(19) United States
(12) Patent Application Publication
(10) Pub. No.: US 2008/0038227 A1
(43) Pub. Date: Feb. 14, 2008

(54) Animal model of neurodegenerative diseases, the procedure for producing the model and applications thereof

(76) Inventors: Ignacio Torres Aleman, Madrid (ES); Eva Maria Carro Diaz, Madrid (ES); Jose Luis Trejo Perez, Madrid (ES); Carlos Spuch Calvar, Madrid (ES); Delphine Bohl, Paris (FR); Jean-Michel Heard, Paris (FR)

Correspondence Address: WENDEROTH, LIND & PONACK, L.L.P., 2033 K STREET N. W., SUITE 800, WASHINGTON, DC, 20006-1021, US

(21) Appl. No.: 11/702,072

(22) Filed: Feb. 5, 2007

Related U.S. Application Data
(63) Continuation-in-part of application No. PCT/ES05/70106, filed on Jul. 21, 2005.
(63) Continuation-in-part of application No. PCT/EP05/13022, filed on Nov. 18, 2005.

(30) Foreign Application Priority Data
Aug. 4, 2004 (ES) P200401946
Nov. 18, 2004 (CA) 2,488,113

(51) Int. Cl. A01K 67/027; A61K 31/7088; A61K 48/00; C12N 15/86

(52) U.S. Cl. 424/93.2; 435/320.1; 514/44; 800/21; 800/23; 800/9

(57) ABSTRACT

The present invention relates to the field of diseases, such as Alzheimer's disease, where abnormal brain accumulation of β amyloid and/or amyloid plaques are involved. More specifically, the present invention relates to a non-human animal model for such diseases and its use in screening methods for molecules for treating same.

### FIELD OF THE INVENTION

The invention is related, in general, to the treatment of neurodegenerative diseases and, in particular, with the development of non-human animals useful as models of neurodegenerative diseases.

### BACKGROUND OF THE INVENTION

The development of experimental models of neurological diseases is of major importance for biomedical research (Cenci M A, Whisaw I Q, Schallert T (2002) Animal models of neurological deficits: how relevant is the rat? Nat Rev Neurosci 3: 574-579). For the case of the neurodegenerative diseases, the development of models nearing the characteristics of the disease in human beings has mean a major methodological advancement. However, on all thereof being produced by conventional genetic engineering, the economic and the personnel and facility-related resources necessary are usually quite large-scale. Although, despite this, the use thereof is becoming widespread, the high cost thereof is generating a tremendous load on the resources devoted to research.

Alzheimer's disease, a typical case of neurodegenerative disease presenting dementia, is the fourth-ranked cause of death in the industrialized countries, with around 13 million individuals affected, a number which could be even greater due to approximately 25% of cases not being diagnosed. The prognosis for the upcoming years is a spiraling rise in the number of those affected, which could exceed 40 million in the industrialized countries where the population is found to be aging (Dekosky et al. (2001) Epidemiology and Pathophysiology of Alzheimer's disease, Clinical Cornerstone 3 (4): 15-26). There are currently few medications effective for treating Alzheimer's disease, and the cost of the treatment of this disease per patient is currently quite expensive, being estimated at around US $225,000, according to data from the American Alzheimer Association. The existence of this serious health problem with a highly limited number of useful medications has prompted research aimed to ascertaining the etiopatogenic mechanism of said neurodegenerative disease for the purpose of identifying and evaluating potentially therapeutic compounds to combat this disease. In the case of Alzheimer's disease, one of the main advancements has come precisely on being able to identify the proteins involved in the familial Alzheimer's disease, which is not associated with aging as is sporadic Alzheimer's disease, which is, by far, the most frequent form of this disease (Mayeux R (2003) Epidemiology of neurodegeneration. Annu Rev Neurosci 26: 81-104).

Transgenic models which are carriers of the different mutations found in familial Alzheimer's disease patients, such as presenilins and amyloid beta (Hock B J, Jr., Lamb B T (2001) transgenic mouse models of Alzheimer's disease. Trends Genet 17: S7-12). One highly important drawback is that although these mutant animals have several symptoms of Alzheimer's disease, none of them shows the full spectrum of pathological changes associated with this disease (Richardson J A, Burns D K (2002) Mouse models of Alzheimer's disease: a quest for plaques and tangles. ILAR J 43: 89-99). In an attempt to solve this problem, transgenic mouse strains with the different mutations which each recreate different aspects of the disease have been crossed with one another in order to thus achieve a model which better resembles the human pathology (Phinney A L, Home P, Yang J, Janus C, Bergeron C, Westaway D (2003) Mouse models of Alzheimer's disease: the long and filamentous road. Neurol Res 25: 590-600). For example, crossing mice which express major amounts of one of the mutated forms of the precursor protein of human amyloid beta (APP-Swe695) with mice which express mutated forms of presenilins generate hybrids which has amyloid plaques along with neurofibrillary tangles and cognitive deficits (Duff K, Eckman C, Zehr C, Yu X, Prada CM, Perez-tur J, Hutton M, Buee L, Harigaya Y, Yager D, Morgan D, Gordon M N, Holcomb L, Refolo L, Zenk B, Hardy J, Younkin S (1996) Increased amyloid-beta42(43) in brains of mice expressing mutant presenilin 1. Nature 383: 710-713; Richards J G, Higgins G A, Ouagazzal A M, Ozmen L, Kew J N, Bohrmann B, Malherbe P, Brockhaus M, Loetscher H, Czech C, Huber G, Bluethmann H, Jacobsen H, Kemp J A (2003) PS2APP Transgenic Mice, Coexpressing hPS2mut and hAPPswe, Show Age-Related Cognitive Deficits Associated with Discrete Brain Amyloid Deposition and Inflammation. J Neurosci 23: 8989-9003).

In following, an indication is provided, for illustrative purposes, of some of the patents related to animal models of Alzheimer's disease: US20030229907, Transgenic non-human mammals with progressive neurologic disease; US20030167486, Double transgenic mice overexpressing human beta secretase and human APP-London; US20030145343, Transgenic animals expressing human p25; US20030131364, Method for producing transgenic animal models with modulated phenotype and animals produced therefrom; US20030101467, Transgenic animal model for Alzheimer disease; US200030093822, Transgenic animal model of neurodegenerative disorders; U.S. Pat. No. 6,717,031, Method for selecting a transgenic mouse model of Alzheimer's disease; U.S. Pat. No. 6,593,512, Transgenic mouse expressing human tau gene; U.S. Pat. No. 6,563,015, Transgenic mice over-expressing receptor for advanced alycation endproduct (RAGE) and mutant APP in brain and uses thereof; U.S. Pat. No. 6,509,515, Transgenic mice expressing mutant human APP and forming congo red staining plaques; U.S. Pat. No. 6,455,757, Transgenic mice expressing human APP and TGF-beta demonstrate cerebrovascular amyloid deposits; U.S. Pat. No. 6,452,065, Transgenic mouse expressing non-native wild-type and familial Alzheimer's Disease mutant presenilin 1 protein on native presenilin 1 null background; WO03053136, Triple transgenic model of Alzheimer disease; WO03046172, Disease model; U.S. Pat. No. 6,563,015, Transgenic mice over-expressing receptor for advanced glycation endproduct (RAGE) and mutant APP in brain and uses thereof; WO0120977, Novel animal model of Alzheimer disease with amyloid plaques and mitochondrial dysfunctions; EP1285578, Transgenic animal model of Alzheimer's disease.

At present, these transgenic animal models are the only ones accepted for the study of pathogenic mechanisms of Alzheimer's disease and for the screening, at the pharmaceutical level, of new drugs. Given the disparity of models which have been generated for the purpose of recreating and analyzing each one of the possible causes of this disease, the availability thereof is restricted in many cases due to property right-related questions and, above all, due to the lack of material resources necessary for generating complex hybrids (Oddo S, Caccamo A, Shepherd J D, Murphy M P, Golde T E, Kayed R, Metherate R, Mattson M P, Akbari Y, LaFerla F M (2003) Triple-transgenic model of Alzheimer's disease with plaques and tangles: intracellular Aβ and synaptic dysfunction. Neuron 39: 409-421). This means severe limitations on the widespread use of these models.

Additionally worthy of special mention is the fact that the mediations currently existing for the treatment of Alzheimer's disease are not very effective and that the models based on existing transgenic animals have deficiencies on not being a true reflection of the pathology of Alzheimer's disease. Therefore, a serious health problem continues to exist with a highly limited number of useful medications, the need therefore existing of developing experimental models alternative to the existing ones which afford the possibility of studying the etiopathogenic mechanism of said neurodegenerative disease and/or of identifying and evaluating potentially therapeutic compounds to combat said disease.

On the other hand, the growth factor receptor similar to Type I insulin (IGF-1) is a membrane protein pertaining to the family of receptors with tyrosin-kinase enzymatic activity, quite similar to the insulin receptor (Ullrich A, Gray A, Tam A W, Yang-Feng T, Tsubokawa M, Collins C, Henzel W, Le Bon T, Kathuria S, Chen E. (1986) Insulin-like growth factor I receptor primary structure: comparison with insulin receptor suggests structural determinants that define functional specificity. EMBO J5: 2503-2512). The ample and highly relevant biological functional have led to its being studied intensively such that the intracellular signaling pathway is relatively well-known (LeRoith D, Werner H, Beitner-Johnson D, Roberts C T, Jr. (1995) Molecular and cellular aspects of the insulin-like growth factor I receptor. Endocr Rev 16:143-163). The role thereof in pathologies such as cancer, diabetes and neurodegeneration were on target in the search for pharmacological modulators of clinical use, although the etiopathogenic role is not know, in pathologies such as Alzheimer's disease, which the functional alteration thereof may induce.

### SUMMARY OF THE INVENTION

The invention confronts the problem of providing new animal models of human neurodegenerative diseases, such as human neurodegenerative diseases which present dementia, one of which is Alzheimer's disease.

The solution provided by this invention is based on the inventors having observed that the repression of the functional activity of the IGF-1 receptor in the epithelial cells of the choroid plexa of the ventricles of an animal's brain makes the development of an animal model of neurodegenerative diseases possible, in general and in particular, an animal model of human neurodegenerative diseases which present with dementia, such as Alzheimer's disease, which fulfills the main characteristics of said human disease, which is simple to produce and which can be used in laboratory animals with any genetic background. For this purpose, and among other technical possibilities, a vector containing a mutated form of the IGF-1 receptor which nullifies the functional activity of this trophic factor at the level of the choroid plexus on serving as a negative dominant (Example 1) was injected by means of stereotaxic surgery into the lateral ventricles of the brain. A few months later, the animal showed all of the symptoms associated with Alzheimer's disease: accumulation of amyloid peptide in the brain, hyperphosphorylated tau protein deposits in conjunction with ubiquitin, loss of synaptic proteins and severe cognitive deficits (learning and memory). The development of the Alzheimer-type pathology appears 3-6 months following the injection of the vector, depending upon the genetic background of the host animal, such that in the genetically-engineered animals which can potentially modulate the onset of Alzheimer's disease, the standard neuropathology of said disease appears earlier (Examples 2 and 3).

Therefore, in one aspect, the invention is related to a non-human animal useful as an experimental model characterized in that it shows an alteration in the biological activity of the IGF-1 receptor located in the epithelial cells of the choroid plexus of the cerebral ventricles. Said non-human animal is useful as an experimental model of neurodegenerative diseases, particularly human neurodegenerative disease which present with dementia, such as Alzheimer's Disease.

In another aspect, the invention is related to a procedure for the production of said non-human animal useful as an experimental model which includes the repression of the functional activity of the IGF-1 receptor in the epithelial cells of the choroid plexus of said non-human animal by means of a transgenesis process. For this purpose, it is necessary for gene structures and vectors to be developed, which, in conjunction with the applications thereof, constitute additional aspects of the present invention.

In another aspect, the invention is related to the use of said non-human animal as an experimental model for the study of the etiopathogenic mechanism of a neurodegenerative disease or for the identification and evaluation of therapeutic compounds to combat said disease. In one particular embodiment, said neurodegenerative disease is a human neurodegenerative disease which presents with dementia, such as Alzheimer's disease.

One of the advantages of the experimental model developed by this invention lies in that it is a perfectly true reflection of the pathology of Alzheimer's disease, as a result of which said model is a qualitative leap forward in the study of the etiopathogenic mechanism of said neurodegenerative disease as well as in the development of effective tools for the identification and evaluation of therapeutic compounds to combat said disease.

On the other hand, the growth factor receptor similar to Type I insulin (IGF-1) is a membrane protein pertaining to the family of receptors with tyrosin-kinase enzymatic activity, quite similar to the insulin receptor (Ullrich A, Gray A, Tam A W, Yang-Feng T, Tsubokawa M, Collins C, Henzel W, Le Bon T, Kathuria S, Chen E. (1986) Insulin-like growth factor I receptor primary structure: comparison with insulin receptor suggests structural determinants that define functional specificity. EMBO J5: 2503-2512). The ample and highly relevant biological function have led to its being studied intensively such that the intracellylar signaling pathway is relatively well-known (LeRoith D, Werner H, Beitner-Johnson D, Roberts C T, Jr. (1995) Molecular and celluylar aspects of the insulin-like growth factor I receptor. Endocr Rev 16: 143-163). The role thereof in pathologies such as cancer, diabetes and neurodegeneration were on target in the search for pharmacological modulators of cinical use, although the etiopathogenic role is not known, in pathologies such as Alzheimer's disease, which the functional alteration thereof may induce.

Alzheimer<'>s disease (AD) is becoming one of the most frequent diseases in modern societies probably due to a longer life-span brought about by medical and societal advances. Studies with familial forms of the disease determined that brain accumulation of amyloid peptides, a hallmark of the disease, is probably the single most important pathogenic event in AD. Despite being the subject of intense scrutiny, the mechanisms underlying abnormal brain accumulation of β amyloid (Aβ) are not yet elucidated. However, the therapeutic benefit of the reduction of amyloid load is now well established<3>. Preventing brain amyloidosis may therefore lead to erradication of AD, a goal that currently appears unattainable.

There is therefore a need in the art for new tools in the discovery of molecules in the prevention and treatment of diseases, such as Alzheimer's disease, where abnormal brain accumulation of β amyloid and/or amyloid plaques are involved. There is also a need to provide for new sceening and treating methods with regards to such diseases.

### BRIEF DESCRIPTION OF THE FIGURES

FIG. 1 is a photo showing that the HIV/GFP lentiviral vector allows the expression of the transgene in the choroid plexus cells, the expression of green fluorescent protein GFP (green) being seen in cells of the choroid plexus (arrows) of adult rat following intracerebroventricular (icv) injection of the HIV/GFP vector. The photo shows sells of the choroid plexus of an animal which was administered, three months prior to be sacrificed, one single icv injection of the HIV/GFP lentiviral vector.

FIG. 2 shows that the administration of the HIV/IGF-IR.KR (HIV/KR) vector to epithelial cells in culture taken from the choroid plexus of postnatal rats generates a loss of response to the IGF-1. Only in cells infected with KR (HIV+KR+ and HIV+KR+IGF+Aβ), but not in those transfected with a null HIV vector (HIV) the IGF-1 does not promote transcytosis of peptide Aβ-40. *P<0.05 vs. all of the other groups.

FIG. 3 shows that the learning (A) and the spatial memorization (B) are decreased in HIV/IGF-IR.KR rats, given that the latter learn more slowly and worse than the control rats (HIV) in the Morris test, consisting of memorizing the position of a platform covered with water in a pool where the animal swims without being able to rest anywhere else but on the platform. HIV/IGF-IR.KR rats: r²=0.8516 vs. control rats r²=0.9884, *P<0.05.

FIG. 4 shows the Aβ levels in cerebral cortex (A) and in cerebrospinal fluid (CSF) (B) of rats injected with the HIV/IGF-IR-KR (HIV/KR) vector. Whilst an increase in produced in the cerebral levels of Aβ, there is a parallel decrease in the CSF, indicating a decrease in the Aβ clearance. The levels were determined by immunoblot densitometry using anti-Aβ antibodies. Representative immunoblots are shown. Levels of calbindin, a neuronal protein, are also evaluated to show the differences are not due to the amount of total protein in each experimental group. *P<0.05 vs. control (rats injected with null HIV).

FIG. 5 shows the levels of hyperphosphorolyated tau (HPF-tau) in the cerebral cortex of rats injected with the HIV7IGF-IR-KR (HIV/KR) vector. FIG. 5a shows the levels of HPF-tau in the cerebral cortex of rats injected with the HIV/IGF-IR.KR (HIW7KR) vector and with the control vector (HIV-control). The levels were determined by immunoblot densitometry with anti-HPF-tau antibodies. *P<0.05 vs. control. FIG. 5B shows the results of a confocal microscopy analysis of the tissular location of the HPT-tau deposits. The HIV/IGF-IR-.KR (HIV/KR) animals (right panel), but not the control animals (treated with HIV, left panel), show accumulations of HPF-tau (red) both inside (arrow) and outside (asterisk) of the neurons (immunopositive for beta-tubulin, in green) in areas of the telencephalon. The yellow-red intracellular signal is revealing of the colocalization of HPF-tau in neurons. FIG. 5C shows that the extracellular accumulations of HPF-tau also contain ubiquitin. A colocalization (yellow accumulations, arrow) of HPF-tau deposits (red) with ubiquitin (green) is produced. The control animals do not have these deposits (data not shown).

FIG. 6 shows a standard Alzheimer neuropathology in mice with modified genetic background. FIG. 6A shows that the old (over 15 months) LID mice treated with the HIV/IGF-IR.KR (HIV/KR) [LID-HIV/IGF-IR.KR) vector practically did not learn the Morris test. Whilst the old or LID mice which were administered only the control viral vector [LID-HIV] learned and retained what they had learned. Similarly, the LID-HIV/IGF-IR.KR mice, where the signaling of the IGF-1 receptor in the choroid plexus has been eliminated, learn significantly worse (*P<0.001 vs. controls). LID-HIV/IGF-IR.KR (n=5): r²=0.6320, LID-HIV (n=7): r²=0.7379; Controls of the same age (n=6), r²=0.7909. FIG. 6B shows that the LID-HIV/IGF-IR.KR animals show accumulations of Aβ, marked with asterisks on the zoom panel) in telecephalon areas which are barely found in the LID-HIV control mice (lower panel).

FIG. 7 Blockade of IGF-I signaling in the choroid plexus. a, HlV-mediated expression of a DN-IGF-IR (KR) blocks IGF-I signaling on cultured choroid plexus epithelial cells. Infected cells do not respond to IGF-I as determined by absence of IGF-l-induced phosphorylation of IGF-IR (pTyrIGF-IR, two viral dilutions tested) and of its downstream kinase Akt (pAkt). Total levels of IGF-IR and Akt remained unaltered. Blots representative of 3 experiments are shown, b, Blockade of IGF-IR in choroid plexus cells results in inhibition of IGF-I-induced albumin transcytosis across the cell monolayer. Representative blot and densitometry histograms are shown. n=3; **p<0.01 vs albumin only, c, GFP expression 3 months after a single icv injection of HIV-GFP. Left: low magnification micrograph depicting GFP expression at the injection site including the choroid plexus of the lateral ventricle and periventricular ependyma; Right: higher magnification micrograph to illustrate GFP expression in choroid plexus cells. A representative rat is shown (n=6). CP, choroid plexus, LV, lateral ventricle, d-f, In vivo IGF-IR blockade after icv delivery of HIV-KR abrogates IGF-I signaling on choroid plexus, d, lntracarotid injection of IGF-I to intact rats results in increased pAkt staining in the choroid plexus. Left: photomicrographs showing pAkt staining in choroid plexus epithelial cells of saline injected (left) and IGF-I injected rats (right). Blot: levels of pAkt are increased after IGF-I. This experiment was done in 3 rats, e, Eight weeks after KR-injection, pAkt levels are no longer increased in the choroid plexus in response to intracarotid IGF-I, as compared to void-vector injected rats (Control). n=3; *p<0.05 vs control+IGF-I. f, On the contrary, the pAkt response to intracerebral IGF-I is preserved after KR administration. pAkt levels were measured in hippocampal tissue surrounding the injection site. Total Akt levels are shown in lower representative blots. n=3; **p<0.01 vs IGF-l-treated. groups g, Passage of intracarotid injected digoxigenin-labelled (DIG) IGF-I into the CSF is blocked 8 weeks after icv injection of KR to adult rats. Representative blot and densitometry histograms. n=3; **p<0.01 vs control.

FIG. 8 Alzheimer's-like neuropathology after in vivo blockade of IGF-IR. a, Western blot analysis with a pan-specific anti-Aβ antibody shows increased Aβ levels in cortex (left) and decreased in CSF (right) after 3 and 6 months of KR injection. Representative blots and densitometry histograms are shown. Controls n=13, three months n=6; six months n=7; *p<0.05 and <**>p<0.01 vs controls, b, ELISA analysis of cortical tissue of KR-injected rats after 6 months shows increases in Aβ 1-40, while Aβ 1-42 remains unchanged. n=7; **p<0.01. c, Parallel decreases in brain (cortex, upper panels) and CSF levels (lower panels) of Aβ carriers such as albumin (left), transthyretin (middle) and apolipoprotein J (apoJ, right) are found 3/6 months after KR. Number of animals as in panel a; *p<0.05 and **p<0.01 vs controls, d, Cognitive deterioration in KR-treated rats is evident at 3 (triangles) and 6 (squares) months after the injection as determined in the water maze test. Both the acquisition (learning) and the retention (memory) phases of the test were affected. *p<0.05 vs KR at 3 and 6 months. Controls (rhombus) n=13; KR three months n=6; six months n=7.

FIG. 9 Alzheimer's-like neuropathology after in vivo blockade of IGF-IR.

a, Levels of dynamin 1 and synaptophysin in cortex are decreased 6 months after KR, while those of GFAP are increased. Representative blots (left) and densitometry histograms (n=6); *p<0.05 and **p<0.0l vs controls, b, Brain levels of pTyr²¹⁶GSK-3β and pSer⁹GSK-3β are oppositely regulated after 3 months of KR, resulting in an increased ratio of the active form of this tau-kinase. Representative blots and densitometry histograms. N=; *p<0.05 and **p<0.01 vs controls, c, Blockade of IGF-IR in the choroid plexus results in heavy PHF-tau brain immunostaining and significantly higher HPF-tau levels. Left: upper photomicrographs illustrates abundant PHF-tau<+> (red) neuronal (calbindin*, green) profiles in the hippocampus after 6 months of KR injection. Note the sparing of HPF-tau immunostaining in control neurons as well as the presence of occasional extracellular HPF-tau deposits in KR rats. GL, granule cell layer, hi, hylus. Middle: Thioflavin-S staining of human AD brain and KR-injected rat brain show the presence of tangles (asterisk) in human but not rat sections. Lower: PHF-tau immunostaining in KR-injected rats and human AD brain sections revealed with diaminobenzydine illustrate the presence of similar intracellular deposits. Right: levels of PHF-tau are increased in the brain of KR-injected rats 3/6 months later. Representative blots and densitometry analysis. Levels of tau remained unaffected (lower blot). n=6; *p<0.05 and **p<0.01 vs controls, d, left: As determined by confocal analysis, PHF-tau (red) deposits co-localize with ubiquitin (green) and are surrounded (right panels) by abundant astrocytic (GFAP⁺, green) profiles. Note the absence of tauopathy in void vector-injected animals (control). Cortical sections are shown.

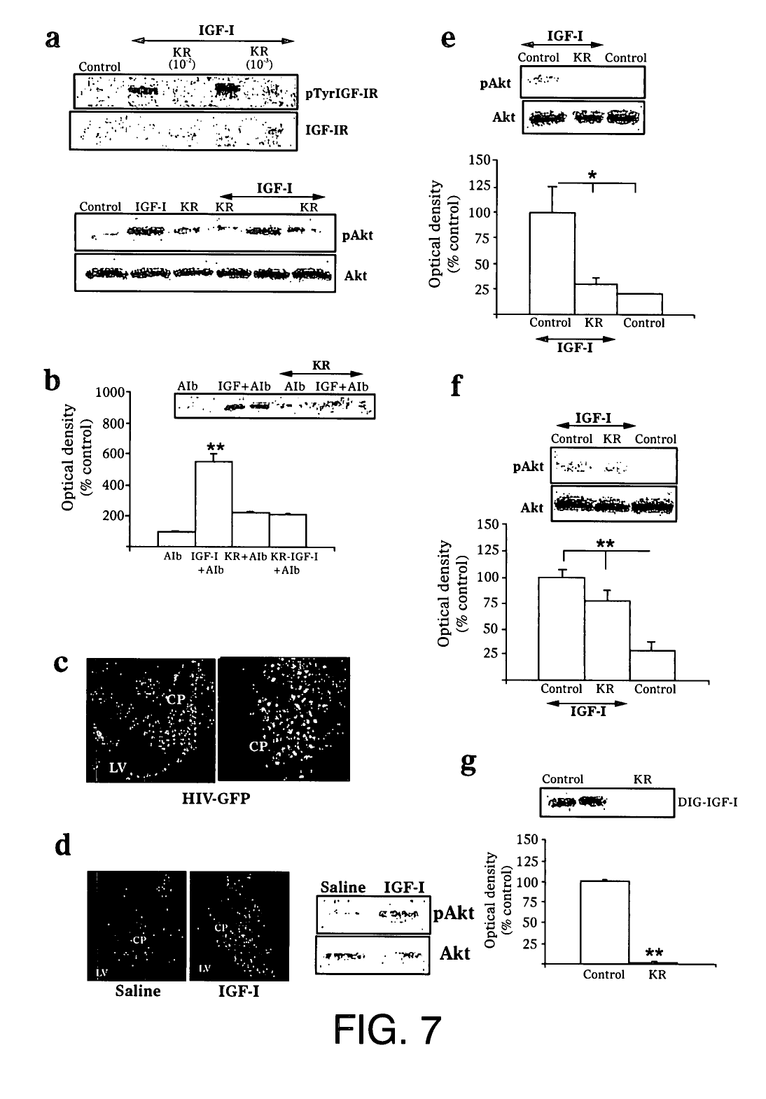

FIG. 10 Restoring IGF-IR function in the choroid plexus reverts most, but not all AD-like disturbances. a, Injection of HIV-wild type (wt) IGF-IR to rats that received HIV-KR 3 months before resulted in normalization of choroid plexus responses to IGF-I. After ic injection of IGF-I, KR-wtlGF-IR treated rats show control pAkt levels in choroid plexus (compare this response to that shown by KR rats in FIG. 7e, n=7). b, However, while memory (retention) scores in the water-maze were also normalized after restoring IGF-IR function, learning (acquisition) the location of the platform remained impaired. N=12 controls (rhombus), n=7 KR-wt-IGF-IR (squares), and n=6 KR-treated groups (triangles); **p<0.01 vs controls, c, On the contrary, levels of brain Aβ₁₋₄₀ were normalized by wtIGF-IR coexpression with KR. N=7 for all groups; *p<0.01 vs controls.

FIG. 11 Exacerbation of AD-like pathology by KR administration to old mutant mice. a, Spatial learning and memory in the water maze test is severely impaired in aged LID mice receiving icv KR 3 months before. Note that void vector treated old LID mice show learning impairment similar to age-matched control littermates as compared to young (6 months-old) wild type littermates. N=5 aged-LID-KR injected mice (squares), n=7 aged LID HIV mice (triangles), n=6 aged intact LIDs, n=6 aged littermate mice (rhombus), n=8 young littermate mice (circles), n=6 young LID mice; *p<0.001 vs aged littermates and void-vector LID mice, and <**>p<0.001 vs young mice, b, Levels of Aβ₁₋₄₀ and of Aβ₁₋₄₂, as determined by ELISA, were not significantly elevated in KR-treated old LID mice as compared to old control LlDs. Note that young LID mice already have high Aβ levels as compared to control littermates and that old (>21 months-old) LIDs show even higher levels. N=; *p<0.05 and **p<0.01 vs respective controls, c, Left: old LlD mice treated with KR show scattered small amyloid plaques. Note diffuse amyloid immunostaining in KR animals, absent in controls. Right: amyloid staining in brain sections of LID (left), human AD (center) and APP/PS2 mice (right) reveals the presence of florid plaques only in the two latter, d, Left: Levels of PHF-tau are significantly increased in KR-treated old LID mice. Representative blot and densitometry is shown. n=5 LID-KR; n=7 LID HIV; n=8 littermates (sham); N=; *p<0.05 vs controls. Right: abundant PHF-tau (red) profiles are found in the hippocampus of LID-KR mice as compared to void vector injected LIDs (controls) or littermates (sham). Neurons are stained with βIII tubulin (green). ML, molecular layer.

FIG. 12 Proposed pathogenic processes in sporadic Alzheimer's disease. 1 : Although during normal aging there is a gradual decline in IGF-I input³⁷, an abnormally high loss of IGF-I input in the choroid plexus develops in sporadic AD as a result of genotype/phenotype interactions. 2: Consequently, Aβ clearance is compromised and Aβ accumulates in brain. In parallel, neuronal IGF-I input is impaired through reduced entrance of systemic IGF-I (see FIG. 7e), associated to increased neuronal resistance to IGF-I (unpublished observations). 3: Loss of sensitivity of neurons to insulin¹⁹ is brought about by the combined loss of sensitivity to IGF-I²⁴ and excess Aβ⁴⁶. The pathological cascade is initiated: tau-hyperphosphorylation, synaptic derrangement, gliosis, cell death and other characteristic features of AD neuropathology are triggered by the combined action of amyloidosis and loss of IGF-I/insulin input. More work is needed to ascertain the validity of this proposal since the present data do not allow to distinguish between steps 2 and 3.

FIG. 13 Description of Lentiviral vector expressing IGF-1R: pHIV-IGF1R.

The following digestion pattern (expressed in bp) can be found for the plasmid after extraction from bacteria and incubation with the following restriction enzymes.

EcoR1: 5515+4793+541+43

Pst1: 7472+1728+1692

Pvu2: 2942+2519+1748+938+771+767+645+578

Bgl2+Xba1: 4126+3654+2323+682+66+41.

FIG. 14 Description of Lentiviral vector expressing IGF-1R: pHIV-IGF1R-DN.

The following digestion pattern (expressed in bp) can be found for the plasmid after extraction from bacteria and incubation with the following restriction enzymes.

EcoR1: 5515+4793+541+43

Pst1: 7472+1728+1692

Pvu2: 2942+2519+1748+938+771+767+645+578

Bgl2+Xba1: 4126+3654+2323+682+66+41.

Sequencing: The plasmid region containing mutation in the transgene (lys 1003 or arg 1003) is the region comprised between bases 7700 and 8100 of pHIV-IGF1-DN. For the deposited strain, this region can be sequenced to confirm viability of the microorganism.

### DETAILED DESCRIPTION OF THE INVENTION

An object of the invention concerns a non-human animal used as a model for disease where abnormal brain accumulation of β amyloid and/or amyloid plaques are involved, wherein β amyloid clearance from brain is decreased. Other objects of the invention concern a method for screening a molecule for the treatment of diseases where abnormal brain accumulation of β amyloid and/or amyloid plaques are involved wherein said method comprises administering said molecule to an animal according to the invention during a time and in an amount sufficient for the Alzheimer's disease-like disturbances to revert, wherein reversion of Alzheimer's disease-like disturbances is indicative of a molecule for the treatment of diseases where abnormal brain accumulation of β amyloid and/or amyloid plaques are involved.

The invention also relates to a method for screening a molecule to prevent the disease from occurring, wherein said molecule prevents or postpones Alzheimer's disease-like disturbance.

Still another object of the invention is to provide a method for treating or preventing a disease where abnormal brain accumulation of β amyloid and/or amyloid plaques are involved in a mammal, wherein said method comprises administering to said mammal a molecule capable of increasing [beta] amyloid clearance from brain.

Yet another object of the invention concerns a process for screening an active molecule interacting with the IGF-I receptor which comprises administering said molecule to an animal during a time and in an amount sufficient for Alzheimer's disease-like disturbances to be modulated, wherein reversion of Alzheimer's disease-like disturbances is indicative of a molecule that increases IGF-I receptor activity and wherein appearance of Alzheimer's disease-like disturbances is indicative of a molecule that decreases IGF-I receptor activity.

A further object of the invention concerns gene transfer vectors capable of either expressing a dominant negative IGF-I receptor or a functional IGF-I receptor.

Yet, a further object of the invention concerns the use of the nucleotide sequence encoding the receptor of IGF-I for the treatment of a disease where abnormal brain accumulation of [beta] amyloid and/or amyloid plaques are involved. One aspect of the present invention is related to a non-human animal useful as an experimental model, referred to hereinafter as animal model of the invention, characterized in that it has an alteration in the biological activity of the growth factor receptor similar to Type I insulin (IGF-1) located in the epithelial cells of the choroid plexus of the cerebral ventricles.

As used in the present invention, the term “non-human animal” refers to a non-human mammal of any genetic background, preferably laboratory animals such as rodents, more preferably rats and mice or non-human primates.

As used in the present invention, the term “any genetic background” refers both to a normal non-human animal and to a transgenic non-human animal.

The term “normal”, applied to animal, as used in the present invention, refers to animals having no transgenes which could be involved in the etiopathogenia of neurodegenerative diseases, for example, human neurodegenerative diseases, for example, human neurodegenerative disease which present with dementia, such as Alzheimer's disease.

The term “transgenic”, applied to animal, as used in the present invention, refers to animals which contain a transgene which could be involved in the etiopathogenia of neurodegenerative diseases, for example, human neurodegenerative diseases which present with dementia, such as Alzheimer's disease, and includes, for illustrative purposes without limiting the scope of the present invention, transgenic animals of the following group: LID mice (Yakar S, Liu J L, Stannard B, Butler A, Accili D, Sauer B, LeRoith D (1999) Normal growth and development in the absence of hepatic insulin-like growth factor I. Proc Natl Acad Sci USA 96: 7324-7329) transgenic animals carriers of mutations in presenilins and beta amyloid (Hock B J, Jr., Lamb B T (2001) Transgenic mouse models of Alzheimer's disease. Trends Genet 17: S7-12), animals carriers of other mutations and alterations (US20030229907, Transgenic non-human mammals with progressive neurologic disease; US20030145343, Transgenic animals expressing human p25; US20030131364, Method for producing transgenic animal models with modulated phenotype and animals produced therefrom; US20030101467, Transgenic animal model for Alzheimer disease; US200030093822, Transgenic animal model of neurodegenerative disorders; U.S. Pat. No. 6,717,031, Method for selecting a transgenic mouse model of Alzheimer's disease; U.S. Pat. No. 6,593,512, Transgenic mouse expressing human tau gene; U.S. Pat. No. 6,563,015, Transgenic mice over-expressing receptor for advanced alycation endproduct (RAGE) and mutant APP in brain and uses thereof; U.S. Pat. No. 6,509,515, Transgenic mice expressing mutant human APP and forming congo red staining plaques; U.S. Pat. No. 6,455,757, Transgenic mice expressing human APP and TGF-beta demonstrate cerebrovascular amyloid deposits; U.S. Pat. No. 6,452,065, Transgenic mouse expressing non-native wild-type and familial Alzheimer's Disease mutant presenilin 1 protein on native presenilin 1 null background; WO03053136, Triple transgenic model of Alzheimer disease; WO03046172, Disease model; U.S. Pat. No. 6,563,015, Transgenic mice over-expressing receptor for advanced glycation endproduct (RAGE) and mutant APP in brain and uses thereof; WO0120977, Novel animal model of Alzheimer disease with amyloid plaques and mitochondrial dysfunctions; EP1285578, Transgenic animal model of Alzheimer's disease) and transgenic animals produced by way of the crossing of strains of transgenic mice with the different mutations which take place in Alzheimer's disease (Phinney A L, Home P, Yang J, Janus C, Bergeron C, Westaway D (2003) Mouse models of Alzheimer's disease: the long and filamentous road. Neurol Res 25: 590-600; Duff K, Eckman C, Zehr C, Yu X, Prada C M, Perez-tur J, Hutton M, Buee L, Harigaya Y, Yager D, Morgan D, Gordon M N, Holcomb L, Refolo L, Zenk B, Hardy J, Younkin S (1996) Increased amyloid-beta42(43) in brains of mice expressing mutant presenilin 1. Nature 383: 710-713; Richards J G, Higgins G A, Ouagazzal A M, Ozmen L, Kew J N, Bohrmann B, Malherbe P, Brockhaus M, Loetscher H, Czech C, Huber G, Bluethmann H, Jacobsen H, Kemp J A (2003) PS2APP Transgenic Mice, Coexpressing hPS2mut and hAPPswe, Show Age-Related Cognitive Deficits Associated with Discrete Brain Amyloid Deposition and Inflammation. J Neurosci 23: 8989-900; US20030167486 Double transgenic mice overexpressing human beta secretase and human APP-London).

The alteration of the biological activity of the IGF-1 receptor function in the epithelial cells of the choroid plexus of the cerebral ventricle of the animal model of the invention will consist, in general, of the functional repression of the biological activity thereof (biological repression).

Said alteration of the biological activity of the IGF-1 receptor function in the epithelial cells of the choroid plexus of the cerebral ventricles may be due to a repression of the functional activity of the IGF-I receptor due to the expression of a polynucleotide the sequence of nucleotides of which encodes a dominant non-functional mutated form of the IGF-I receptor. In one particular embodiment, said polynucleotide encodes a dominant non-functional mutated form of the human IGF-I receptor. For illustrative purposes, said dominant non-functional mutated form of the human IGF-1 receptor is selected between the nonfunctional mutated form of the IGF-1 receptor referred to as IGF-IR.KR, which has the K1003R mutation, in which the lysine residue of position 1003 of the amino acid sequence of the human IGF-I receptor has been substituted for an arginine residue and the nonfunctional mutated form of the IGF-I receptor referred to as IGF-IR.KA, which has the K1003A mutation, in which the lysine residue in position 1003 of the amino acid sequence of the human IGF-I receptor has been substituted for an alanine residue (Kato H, Faria T N, Stannard B, Roberts C T, Jr., LeRoith D (1993) Role of tyrosine kinase activity in signal transudation by the insulin-like growth factor-I (IGF-I) receptor. Characterization of kinase-deficient IGF-I receptors and the action of an IGF-I-mimetic antibody (alpha IR-3). J Biol Chem 268: 2655-2661). The numbering system used for numbering the amino acid residues of the human IGF-I receptor is that used by Ullrich et al. (Ullrich A. et al. 1985) Human insulin receptor and its relationship to the tyrosine kinase family of oncogenes. Nature 313:756-761; Ullrich A. et al. (1986) Insulin-like growth factor I receptor primary structure: comparison with insulin receptor suggests structural determinants that define functional specificity. EMBO J. October 1986; 5(10): 2503-2512).

Alternatively, said alteration of the biological activity of the IGF-I receptor function in the epithelial cells of the choroid plexus of the cerebral ventricles can be due to the repression of the functional activity of the IGF-I receptor due to the expression of a polynucleotide the sequence of nucleotides of which encodes an element inhibiting the expression of the gene of the IGF-I receptor capable of repressing the functional activity thereof. As used in the present invention, the term “element inhibiting the expression of the IGF-I receptor gene capable of repressing the functional activity thereof” refers to a protein, enzymatic activity or sequence of nucleotides, RNA or DNA, single or double-strand, which inhibits the translation into protein of the mRNA of the IGF-I receptor. For illustrative purposes, said polynucleotide can be a polynucleotide which encodes a specific sequence of antisense nucleotides of the sequence of the gene or of the mRNA of the IGF-I receptor, or rather a polynucleotide which encodes a specific aptamer of the mRNA of the IGF-I receptor, or rather a polynucleotide which encodes a specific interference RNA (“small interference RNA” or siRNA) of the mRNA of the IGF-I receptor.

The animal model of the invention can have any genetic background; nevertheless, in one particular embodiment, said animal model of the invention comes from a normal animal, advantageously, from a healthy normal animal, in other words, which has no diagnosed pathology, such as a healthy rat (Example 2), whilst in another particular embodiment of the invention, it comes from a transgenic animal, such as an LID transgenic mouse (Example 3).

The animal model of the invention is an animal useful as an experimental model of neurodegenerative diseases, for example, neurodegenerative diseases which present with dementia. Preferably, said neurodegenerative diseases are human neurodegenerative diseases, more preferably human neurodegenerative diseases which present with dementia. In one particular embodiment, said human neurodegenerative disease which presents with dementia is Alzheimer's disease. Alzheimer's disease totals 60% of the dementia cases, whilst microvascular or multi-infarct disease totals 20% thereof. Other minor causes of dementia are chronic alcohol and drug abuse and very low-incidence neurological disease, such as Pick's disease and Creutzfeldt-Jacob disease.

Therefore, in another aspect, the invention is related to the use of the animal model of the invention as an experimental model of neurodegenerative diseases, such as neurodegenerative diseases which present with dementia; preferably, said neurodegenerative diseases are human neurodegenerative diseases, such as human neurodegenerative diseases which present with dementia; for example, Alzheimer's disease.

Likewise, the use of the animal model of the invention for the study of the etiopathogenic mechanisms of neurodegenerative diseases, particularly human neurodegenerative diseases and, more particularly, human neurodegenerative diseases which present with dementia, such as Alzheimer's disease, as well as the use of the animal model of the invention for the identification and evaluation of potentially therapeutic compounds to combat said diseases constituting additional aspect of the present invention.

The animal model of the invention can be produced by means of a transgenesis process allows the functional repression of the IGF-I receptor in the epithelial cells of the choroid plexus of said animal model of the invention.

Therefore, in another aspect, the invention is related to a procedure for the production of the animal model of the invention, referred to hereinafter as the procedure of the invention, which includes the repression of the functional activity of the IGF-I receptor of the epithelial cells of the choroid plexus of said animal model of the invention by means of a transgenesis process.

As used in the present invention, the term “transgenesis process” refers to any technique or procedure which permits the integration of an exogenous gene or “transgene” into a series of cells of a live organism without affecting al of the cells of said organism, and which confers a new biological property upon said cells and upon the organism carrying the same. Said transgene or exogenous gene refers to a DNA normally not resident or present in the cell which is aimed at being transformed.

On the other hand, the transgenesis process for producing the animal model of the invention can be applied both to fully-developed animals and to embryos thereof provided that it permit the repression of the functional activity of the IGF-I receptor in the epithelial cells of the choroid plexus of said fully-developed animal model.

In one particular embodiment, said transgenesis process which leads to the repression of the functional activity of the IGF-I receptor includes the transformation of epithelial cells of the choroid plexus of a fully-developed non-human animal such that they express a dominant non-functional mutated form of the IGF-I receptor. This objective can be achieved by means of the administration to epithelial cells of the choroid plexus of said non-human animal of a gene structure which includes a polynucleotide the nucleotide sequence of which encodes a dominant non-functional mutated form of the IGF-I receptor for the purpose of transforming said epithelial cells of the choroid plexus so that they will express said dominant non-functional mutated form of the IGF-I receptor. Advantageous, said gene structure is included within a vector, such as, for example, an expression vector or a transference vector.

As used in the present invention, the term “vector” refers to systems utilized in the transference process of an exogenous gene or of an exogenous gene structure to the inside of a cell, thus permitting the stable vehiculation of genes and exogenous gene structures. Said vectors can be non-viral vectors or viral vectors, preferably viral vectors given that the transgenesis with viral vectors has the advantage of being able to direct the expression of a foreign gene in adult tissues relatively precisely and is one of the reasons why the general use thereof for gene therapy is being posed (Pfeifer A, Verma I M (2001) Gene therapy: promises and problems. Annu Rev Genomics Hum Genet 2: 177-211).

The invention has been exemplified by means of the use of lentiviral vectors. These vectors are easy to handle, one of the main advantages thereof being their effective transduction, their genomic integration and their persistent or prolonged expression. Other appropriate viral vectors include retroviral, adenoviral or adenoassociated vectors (Consiglio A, Quattrini A, Martino S, Bensadoun J C, Dolcetta D, Trojani A, Benaglia G, Marchesini S, Cestari V, Oliverio A, Bordignon C, Naldini L (2001) In vivo gene therapy of metachromatic leukodystrophy by lentiviral vectors: correction of neuropathology and protection against learning impairments in affected mice. Nat Med 7: 310-316; Kordower J H, Emborg M E, Bloch J, Ma S Y, Chu Y, Leventhal L, McBride J, Chen E Y, Palfi S, Roitberg B Z, Brown W D, Holden J E, Pyzalski R, Taylor M D, Carvey P, Ling Z, Trono D, Hantraye P, Deglon N, Aebischer P (2000) Neurodegeneration prevented by lentiviral vector delivery of GDNF in primate models of Parkinson's disease. Science 290: 767-773). Examples of lentiviral vectors include the type 1 human immunodeficiency virus (HIV-1), of which numerous appropriate vectors have been developed. Other lentiviruses appropriate for their use as vectors include the primate lentivirus group including the type 2 human immunodeficiency virus (HIV-2), the 3 human immunodeficiency virus (HIV-3), the simian immunodeficiency virus (SIV), the simian AIDS retrovirus (SRV-1), the type 4 human T-cell lymphotrophic virus (HTLV4), as well as the bovine lentivirus, equine lentivirus, feline lentivirus, ovine/caprine lentivirus and murine lentivirus groups.

The invention provides a vector, such as a viral vector, specifically a lentiviral vector, useful for producing an animal model of the invention, which is useful as an experimental model of neurodegenerative disease, specifically, as a model of human neurodegenerative diseases which present with dementia, such as Alzheimer's disease. Said vector as well as the production thereof shall be described in greater detail at a further point herein.

The administration of said gene structure which includes a polynucleotide the nucleotide sequence of which encodes a dominant non-functional mutated form of the IGF-I receptor or of said vector which includes said gene structure, to the epithelial cells of the choroid plexus of the non-human animal to be transformed, can be carried out by many conventional method; nevertheless, in one particular embodiment, the administration of said vector to said epithelial cells of the choroid plexus is carried out by means of intracerebroventricular (icv) injection.

As used in the present invention, the term “a dominant non-functional mutated form of the IGF-I receptor” includes any mutated form of the IGF-I receptor which acts as negative dominant by recombination with the endogenous normal IGF-I receptor, repressing the biological function thereof, in the course of the procedure developed b the present invention. Said dominant non-functional mutated form of the IGF-I receptor is expressed by epithelial cells of the choroid plexus of the animal model of the invention as a result of the transformation thereof with a gene structure which includes a polynucleotide the nucleotide sequence of which encodes said dominant non-functional mutated form of the IGF-1 receptor. In one particular embodiment, said polynucleotide encodes a dominant non-functional mutated form of the human IGF-I receptor. In other particular embodiments, said polynucleotide encodes a dominant non-functional mutated form of the IGF-I receptor of an animal species other than human, such as a mammal, for example a rodent or a non-human primate.

Although practically any dominant non-functional mutated form of the IGF-I receptor can be used for the purpose of achieving functional repression of the biological activity (biological repression) of the IGF-I receptor, in one particular embodiment, said dominant non-functional mutated form of the IGF-I receptor is selected among the non-functional mutated forms of the human IGF-I receptor known as IGF-IR.KR and IGF-IR.KA in this description, defined previously.

The non-human animal whose epithelial cells of the choroid plexus of the cerebral ventricles are going to be transformed by means of the administration of the transgene can have any genetic background.

The procedure of the invention is materialized, in one specific embodiment, in a procedure for the production of an animal model of the invention in which the vector utilized is the lentiviral vector of HIV-1 origin known as HIV/IGF-IR.KR (HIV/KR) in this description, the dominant non-functional mutated form of the IFG-I receptor is the nonfunctional mutated form of the human IGF-I receptor known as IGF-IR.KR, and the non-human animal whose choroid plexus epithelial cells have been transformed is a healthy adult normal rat (Example 2).

Additionally, the procedure of the invention is materialized, in another specific embodiment, in a procedure for the production of an animal model of the invention in which the vector utilized is the lentiviral vector of known as HIV/IGF-IR.KR (HIV/KR), the dominant non-functional mutated form of the IFG-I receptor is the nonfunctional mutated form of the human IGF-I receptor known as IGF-IR.KR, and the non-human animal whose choroid plexus epithelial cells have been transformed is a LID transgenic mouse (Example 3).

Alternatively, as previously mentioned hereinabove, the alteration of the biological activity of the function of the IGF-I receptor in the epithelial cells of the choroid plexus of the cerebral ventricles can be due to the repression of the functional activity of the IGF-I receptor due to the expression of a polynucleotide the nucleotide sequence of which encodes an element inhibiting the expression of the IGF-I receptor gene capable of repressing the functional activity thereof.

Therefore, in another particular embodiment, said transgenesis process of repressing the functional activity of the IGF-I receptor includes the transformation of epithelial cells of the choroid plexus of a non-human animal by means of the introduction of a gene structure which includes a polynucleotide the nucleotide sequence of which encodes an element inhibiting the expression of the gene of the IGF-I receptor capable of repressing the biological activity thereof, said inhibiting element being selected among:

a) a specific antisense nucleotide sequence of the gene sequence or of the mRNA of the IGF-1 receptors b) a specific ribosome of the mRNA of the IGF-1 receptor c) a specific aptamer of the mRNA of the IGF-I receptor, or d) a specific interference RNA 8iRNA) of the mRNA of the IGF-I receptor.

Advantageous, said gene structure is included within a vector, such as, for example, an expression vector or a transference vector. The characteristics of said vector have been previously defined.

The aforementioned a)-d) nucleotide sequences prevent the expression of the gene in mRNA or of the mRNA in the protein of the IGF-1 receptor and therefore repress the biological function thereof and can be developed by an expert in the genetic engineering sector in terms of the existing know-how in the state of the art on transgenesis and gene expression repression (Clarke, A. R. (2002) Transgenesis Techniques. Principles and Protocols, 2^{nd} Ed Humana Press, Cardiff University; Patent US20020128220. Gleave, Martin. TRPM-2 antisense therapy; Puerta, Ferández E et al. (2003) Ribozymes: recent advances in the development of RNA tools. FEMS Microbiology Reviews 27: 75-97; Kikuchi, et al., 2003. RNA aptamers targeted to domain II of Hepatitis C virus IRES that bind to its apical loop region. J. Biochem 133, 263-270; Reynolds A. et al., 2004. Rational siRNA design for RNA interference. Nature Biotechnology 22 (3): 326-330).

In another aspect, the invention is related to a vector useful for putting the procedure for producing the animal model of the invention into practice. Said vector can be a non-viral vector or, advantageously, a viral vector, as has been previously mentioned hereinabove, and includes a polynucleotide the nucleotide sequence of which encodes a dominant non-functional mutated form of the IGF-I receptor or rather a polynucleotide the nucleotide sequence of which encodes an element inhibiting the expression of the IGF-receptor gene capable of repressing the functional activity thereof, in conjunction, optionally, with the necessary elements for permitting the expression thereof in cells of non-human animals. Said vectors can be in the form of artificial or chimeric viral particles.

In one particular embodiment, said vector is a lentiviral vector which includes a polynucleotide the nucleotide sequence of which is selected between a sequence of nucleotides which encodes a dominant non-functional mutated form of the IGF-I receptor and a sequence of nucleotides which encodes an element inhibiting the expression of the IGF-I receptor gene capable of repressing the functional activity thereof.

In one particular embodiment, the sequence of nucleotides which encodes a dominant non-functional mutated form of the IGF-I receptor is selected between the nonfunctional mutated forms of the human IGF-I receptor known as IGF-IR.KR and IGF-IR.KA in this description, previously defined.

In another particular embodiment, the sequence of nucleotides which encodes an element inhibiting the expression of the IGT-I receptor gene capable of repressing the functional activity thereof is selected between a sequence which encodes. A) a specific antisense sequence of nucleotides of the gene sequence or of the mRNA of the IGF-I receptor; b) a specific ribosome of the mRNA of the IGF-I receptor; c) a specific aptamer of the mRNA of the IGF-I receptor; and d) a specific interference RNA (iRNA) of the mRNA of the IGF-I receptor.

The invention provides, in one specific embodiment, a lentiviral vector which can be obtained by means of transitory transfection in packaging cells of:

a plasmid (i) which includes a sequence of nucleotides selected between:
a sequence of nucleotides which encodes a dominant non-functional mutated form of the IGF-I receptor, and
a sequence of nucleotides which encodes an element inhibiting the expression of the gene of the IGF-I receptor capable of repressing the functional activity thereof;

a plasmid (ii) which includes the sequence of nucleotides which encodes the Rev protein;

a plasmid (iii) which includes the sequence of nucleotides which encodes the Rev response element (RRE); and

a plasmid (iv) which includes the sequence of nucleotides which encodes the heterologous packaging of the vector.

Although practically any appropriate packaging cell can be used, in one particular embodiment, said packaging cells pertain to the 293T-cell line, a line of commercially available transformed human kidney epithelial cells.

Plasmid (i) is a vector, such as a transference or expression vector, which has a gene structure which includes the transgene in question and a functional promoter in the packaging cells which make it possible for the vector being transcripted to be efficiently generated in the packaging cells. In one particular embodiment, said plasmid (i) includes a sequence of nucleotides which encodes a dominant non-functional mutated form of the IGF-I receptor selected between the non-functional mutated forms of the human IGF-I receptor referred to as IGF-IR.KR and IGF-IR.KA in this description, previously defined. In another particular embodiment, said plasmid (i) includes a sequence of nucleotides which encodes an element inhibiting the expression of the IGF-I receptor gene capable of repressing the functional activity thereof selected between a sequence which encodes: a) a specific sequence of antisense nucleotides of the sequence of the gene or of the mRNA of the IGF-I receptor; b) a specific ribosome of the mRNA of the IGF-I receptor; c) a specific aptamer of the mRNA of the IGF-I receptor; and d) a specific interference RNA (iRNA) of the mRNA of the IGF-I receptor.

Plasmid (ii) is a non-overlapping vector which virtually can contain the sequence of nucleotides which encodes any Rev protein, which promotes the cytoplasmic accumulation of the viral transcribes; nevertheless, in one particular embodiment, said plasmid (ii) is a plasmid identified as RSV-Rev, which includes the sequence of nucleotides which encodes the Rev protein of the Rous sarcoma virus (RSV).

Plasmid (iii) is a condition packaging vector and contains the sequence of nucleotides which encodes any appropriate Rev response element (RRE), to which it is joined such that the gene is expressed and the new viral particles are produced.

Plasmid (iv) contained the sequence of nucleotides which encodes the heterologous vector packaging, as a result of which it can contain the sequence of nucleotides which encodes any protein of the packaging of an appropriate virus, with the condition that said virus not be a lentivirus; nevertheless, in one particular embodiment, said plasmid is that known as p-VSV, which includes the sequence of nucleotides which encodes the packaging of the vesicular stomatitis virus (VSV).

Said lentiviral vector can be produced by conventional methods known by experts on the subject.

In one particular embodiment, said lentiviral vector is referred to as HIV7IGF-IR.KR (HIV/KR) (Example i) which allows the expression of the non-functional mutated form of the IGF-I receptor referred to as IGF-IR-KR which has a K1003R mutation in the amino acid sequence of the human IGF-I receptor, in non-human animal cells and the biological repression of the IGF-I receptor and the development of a non-human animal useful as an experimental model of human neurodegenerative diseases which present with dementia, such as Alzheimer's disease.

In another particular embodiment, said transgenesis process which leads to the repression of the functional activity of the IGF-I receptor in the epithelial cells of the choroid plexus of the animal model of the invention includes a conventional transgenesis process in the embryonic stage of said animal such that the future cells of the choroid plexus of said animal are genetically transformed and lose the capacity to respond to the IGF-I. The development of this type of transgenic animal can be carried out by an expert in the genetic engineering sector in terms of the existing know-how in the state of the art regarding transgenic animals (Bedell M A, Jenkins N A, Copeland N G. Mouse models of human disease. Part I: techniques and resources for genetic analysis in mice. Genes Dev. Jan. 1, 1997; 11(1):1-10. Bedell M A, Largaespada D A, Jenkins N A, Copeland N G. Mouse models of human disease. Part II: recent progress and future directions. Genes Dev. Jan. 1, 1997; 11(1): 11-43).

One possibility of the present invention is a conventional transgenesis procedure by which the expression of a transgene which includes a specific tissue promoter (such as, for example, a transthyretin promoter, Ttr¹ (Schreiber, G. The evolution of transthyretin synthesis in the choroid plexus. Clin. Chem Lab Med. 40, 1200-1210 (2002) and a polynucleotide the sequence of nucleotides of which encodes a dominant non-functional mutated form of the IGF-I receptor. Thus, the dominant non-functional mutated form of the IGF-I receptor solely will be expressed in the cells of the choroid plexus, thus producing the animal model of the present invention. In one particular embodiment, said polynucleotide encodes a dominant non-functional mutated form of the human IGF-I receptor. For illustrative purposes, said dominant non-functional mutated form of the human IGF-I receptor is selected between the non-functional mutated form of the IGF-I receptor referred to as IGF-IR.KR which has the K1003R mutation, in which the lysine residue of the 1003 position of the amino acid sequence of the human IGF-I receptor has been substituted for an arginin residue and the non-functional mutated form of the IGF-I receptor referred to as IGF-IR.KA which has the K1003 mutation, in which the lysine reside of the 1003 position of the amino acid sequence of the human IGF-I receptor has been substituted for an alanin reside (Kato H, Faria T N, Stannard B, Roberts C T, Jr., LeRoith D (1993) Role of tyrosine kinase activity in signal transduction by the insulin-like growth factor-I (IGF-I) receptor. Characterization of kinase-deficient IGF-I receptors and the action of an IGF-I-mimetic antibody (alpha IR-3). J Biol Chem 268: 2655-2661).

Alternatively, said alteration in the biological activity of the IGF-I receptor function in the epithelial cells of the choroid plexus of the cerebral ventricles of said transgenic animals can be produced by the repression of the functional activity of the IGF-I receptor due to the expression of a polynucleotide the sequence of nucleotides of which encodes an element inhibiting the expression of the IGF-I receptor gene capable of repressing the functional activity thereof. As used in the present invention and, as previously stated hereinabove, the term “element inhibiting the expression of the IGF-I receptor gene capable of repressing the functional activity thereof” refers to a protein, enzymatic activity or sequence of nucleotides, RNA or DNA, single or double-strand, which inhibits the translation into protein of the mRNA of the IGF-I receptor. For illustrative purposes, said polynucleotide can be a polynucleotide which encodes a specific sequence of antisense nucleotides of the sequence of the gene or of the mRNA of the IGF-I receptor, or rather a polynucleotide which encodes a specific aptamer of the mRNA of the IGF-I receptor, or rather a polynucleotide which encodes a specific interference RNA (“small interference RNA” or siRNA) of the mRNA of the IGF-I receptor.

Likewise, an animal model of the invention can be produced by conventional transgenesis in which the repression of the functional activity of the IGF-I receptor can be regulated by different mechanisms which would allow for a better control and use of the animal. Thus, one controlled transgenesis technique can consist of the use of the “Cre/Lox” system by means of crossing animals with Lox-IGF-IR (knock-in” systems) transgenic sequences which substitute the endogenous IGF-IR sequence, with animals which have Cre bacterial recombinase controlled by a specific tissue promoter, once again, for example, that of transtyrretin (Isabelle Rubera, Chantal Poujeol, Guillaume Bertin, Lilia Hasseine, Laurent Counillon, Philippe Poujeol and Michel Tauc (2004) Specific Cre/Lox Recombination in the Mouse Proximal Tubule. J Am Soc Nephrol. 15 (8): 2050-6; Ventura A, Meissner A, Dillon C P, McManus M, Sharp P A, Van Parijs L, Jaenisch R, Jacks T. (2004) Cre-lox-regulated conditional RNA interference from transgenes. Proc Natl Acad Sci USA. 101 (28): 10380-5). Another example for generating another controllable transgenic model animal would consist of the use of the “tet-off” system (Rennel E, Gerwins P. (2002) How to make tetracycline-regulated transgene expression go on and off. Anal Biochem. 309 (1): 79-84; Schonig, K. Bujard H. (2003) Generating conditional mouse mutants via tetracycline-controlled gene expression. In: Transgenic Mouse Methods and Protocols, Hofker, M, van Deursen, J (eds.) Humana Press, Totowa, N.J., pages 69-104). One embodiment exemplifying the present invention will consist of a Lox-IFT-IR mouse which is crossed with a Tre-Cre mouse—where Tre is the controllable promoter of the Tta protein (tetracycline-controlled transactivator protein); this hybrid subsequently being crossed with a Ttr-Tta mouse such that the resulting mouse: Lox-IGF-IR/Tre-Cre/Ttr-Tta will eliminate the IGF-IR function in response to the administration of tetracycline, a compound which eliminates the action of the Tta protein.

In another aspect, the invention is related to the use of a vector of the invention in a procedure for the production of a non-human animal useful as an experimental model, such as an experimental model of neurodegenerative disease, particularly human neurodegenerative diseases, especially as a model of human neurodegenerative diseases which present with dementia, such as Alzheimer's disease.

Vectors of the Invention

According to an embodiment of the invention, the present invention is concerned with gene transfer vectors capable of either expressing a dominant negative IGF-I receptor or a functional IGF-I receptor. The gene transfer vectors contemplated by the present invention are preferably derived from HIV or adeno-associated viral (AAV) vectors. Among those vectors that express a dominant negative IGF-I receptor, the present invention preferably consists of the vector deposited at CNCM on Nov. 10, 2004 under accession number 1-3316. Among those vectors that express a functional IGF-I receptor, the present invention preferably consists of the vector deposited at CNCM on Nov. 10, 2004 under accession number 1-3315. As can be appreciated, supplemental informations concerning the vectors of the invention as well as notions on viral vector in general are recited hereafter. pHlV-IGF1R deposited under N[deg.] CNCM 1-3315 is a recombinant plasmid derived from pbr322 encoding the genome of a lentiviral vector which carries a transcription unit having:

the promoter of human phosphoglycerate kinase,

a human cDNA encoding the native form of the receptor for Insulin-Growth factor.

The vector is inserted in E. coli E12 cells which can be cultivated in LB medium with ampicilin. Conditions for seeding are 100 μl in 3 ml LB medium with ampicilin and incubation is carried out at 30° C. under shaking.

The storage conditions are freezing at −80° C. in suspending fluid: Vz bacterial culture (100 μl for 3 ml) and ½ glycerol.

According to the CGG classification the deposited microorganism belongs to Group 2, class 2 and L1 type for confinement.

pHIV-IGF1 R-DN deposited under N° CNCM 1-3316 has the same characteristics as pHIV-IGF1 R except' for the human cDNA that it contains which encodes a negative transdominant mutant of the receptor for Insulin-Growth factor according to Fernandez et al 2001. Genes Dev. 15: 1926-1934.

Non-Human Animal Disease Model

According to another embodiment, the present invention relates to a non-human animal used as a model for disease where abnormal brain accumulation of β amyloid and/or amyloid plaques are involved, wherein β amyloid clearance from brain is decreased. Such a disease preferably contemplated by the present invention is Alzheimer's disease. As used herein, the term “non-human animal” refers to any non- human animal which may be suitable for the present invention. Among those non-human animals, rodents such as mice and rats, and primates such as cynomolgus macaques (Macaca fascicularis) are preferred. The cited animals are examples of animals suitable for use as models, i.e., animals suitable for constituting laboratory animals. The invention is especially directed to such laboratory animals, used or intended for use in research or testing.

According to a preferred embodiment, the IGF-IR function of the animal of the invention is impeded in the choroid plexus epithelium. Even more preferably, the IGF-IR function of the animal is impeded by gene transfer into the choroid plexus epithelial cells with a gene transfer vector as defined above which expresses a dominant negative IGF-I receptor. Preferably, such a vector is the one deposited at CNCM on Nov. 10, 2004 under accession number I-3316.

Therefore, the invention relates especially to non-human transgenic animal wherein gene transfer has been carried out in order to impede the IGF-IR function of the original animal. Accordingly, where reference is made in the present application, to non-human animal suitable for use as disease model, it encompasses such transgenic animals. In a preferred embodiment, a non-human animal suitable for use as disease model specifically corresponds to such transgenic animals.

Methods of Use

According to another embodiment, the present invention provides a method for screening a molecule for the treatment of diseases where abnormal brain accumulation of [beta) amyloid and/or amyloid plaques are involved wherein said method comprises administering said molecule to an animal as defined above during a time and in an amount sufficient for the Alzheimer's disease-like disturbances to revert, wherein reversion of Alzheimer's disease-like disturbances is indicative of a molecule for the treatment of diseases where abnormal brain accumulation of [beta] amyloid and/or amyloid plaques are involved.

By the term “treating” is intended, for the purposes of this invention, that the symptoms of the disease be ameliorated or completely eliminated.

The invention also relates to a method for screening a molecule for preventing a disease (including for preventing its symptoms to arise), where said disease (or symptoms) involve abnormal brain accumulation of [beta] amyloid and/or amyloid plaques, wherein said method comprises administering said molecule to an animal as defined above and detecting if Alzheimer's disease-like disturbances arrive, wherein where if such disturbances do not appear after a period of observation whereas such disturbances appear in the same type of animal during the same period of observation when said same type of animal has not been received said molecule, the molecule is considered to be a candidate to prevent the disease.

The method of screening according to the invention is a method aiming at determining the effect of a test molecule on disturbances induced by or expressed in Alzheimer's disease-like diseases. Accordingly, the screening method of the invention encompasses using an animal as defined in the invention, administering the test molecule to said animal, determining the effet of said test molecule on the disturbances of concern and possibly including at some stage sacrifying the animal. The invention also relates to the use of the animal described according to the invention, as animal model in a screening method for test molecules. The screening method can comprise, in the frame of the determination of the effect of the test molecule on disturbances of concern, brain imaging (e.g., MRI (Magnetic Resonance Imaging), PET scan (Ponction Emission Tomography scan)) and/or behavioral evolution of the animal model and/or in vitro studies on the effects of said test molecules on samples, especially tissue or cell extracts, obtained from said animal.

According to another embodiment, the present invention provides a method for treating a disease, such as Alzheimer's disease, where abnormal brain accumulation of β amyloid and/or amyloid plaques are involved in a mammal, such as a human, wherein said method comprises administering to said mammal a molecule capable of increasing β amyloid clearance from brain. According to a preferred embodiment, the clearance of β amyloid is increased by increasing the activity of IGF-I receptor in choroid plexus epithelial cells. The invention also relates to the use of a test molecule that has shown to improve or revert condition in a patient having Alzheimer's disease-like disturbances in a method of screening of the invention, for the preparation of a drug for the treatment of an Alzheimer or an Alzheimer-like disease. It will be understood that such a molecule contemplated by the present invention preferably promotes the entrance of a protein acting as a carrier of β amyloid through the choroid plexus into the cerebrospinal fluid. Advantageously, the carrier is chosen from albumin, transthyretin, apolipoprotein J or gelsolin.

According to a preferred embodiment, the molecule which is administered to the animal for increasing said IGF-I receptor activity is a gene transfer vector capable of inducing the expression of IGF-I receptor in target cells, such as one as described above and more preferably, the vector deposited at CNCM on Nov. 10, 2004 under accession number I-3315. The molecule to be used in the treating method of the invention is preferably administered to the mammal in conjunction with an acceptable vehicle. As used herein, the expression “an acceptable vehicle” means a vehicle for containing the molecules preferably used by the treating method of the invention that can be administered to a mammal such as a human without adverse effects. Suitable vehicles known in the art include, but are not limited to, gold particles, sterile water, saline, glucose, dextrose, or buffered solutions. Vehicles may include auxiliary agents including, but not limited to, diluents, stabilizers (i. e., sugars and amino acids), preservatives, wetting agents, emulsifying agents, pH buffering agents, viscosity enhancing additives, colors and the like.

The amount of molecules to be administered is preferably a therapeutically effective amount. A therapeutically effective amount of molecules is the amount necessary to allow the same to perform its desired role without causing overly negative effects in the animal to which the molecule is administered. The exact amount of molecules to be administered will vary according to factors such as the type of condition being treated, the mode of administration, as well as the other ingredients jointly administered.

The molecules contemplated by the present invention may be given to a mammal through various routes of administration. For instance, the molecules may be administered in the form of sterile injectable preparations, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intradermalˆ, intramuscularly or sub-cutaneously by injection, by infusion or per os. Suitable dosages will vary, depending upon factors such as the amount of the contemplated molecule, the desired effect (short or long term), the route of administration, the age and the weight of the mammal to be treated. Any other methods well known in the art may be used for administering the contemplated molecule.

In a related aspect and according to another embodiment, the present invention is concerned with the use of the nucleotide sequence encoding the receptor of IGF-I for the treatment or prevention of a disease, such as Alzheimer's disease, where abnormal brain accumulation of β amyloid and/or amyloid plaques are involved.

Reference is made to Ebina Y. et al, 1985 (Cell. Apr, 40(4): 747-58) and Ullrich A. et al (1985 (Nature February 28-March 6, 313 (6005): 756-61) regarding the description of human insulin receptor coding sequence.

The sequence of the human IGF-I is contained as an insert within vector pHIV- IGFIR deposited at the CNCM under N<0> I-3315.

The invention also relates to the use of a nucleotide sequence encoding a polypeptide having a function analogous to the function of the IGF-I receptor, for the prevention or the treatment of a disease where abnormal brain accumulation of β amyloid and/or amyloid plaques are involved, such a nucleotide sequence encoding a polypeptide which is an active fragment of the IGF-1 receptor. An “active fragment” means a polypeptide having part of the amino acid sequence of the IGF-I receptor and which has effect on the regulation of Aβ clearance as disclosed above.

A polypeptide having an analogous function to that of the IGF-1 receptor is a polypeptide similar to said receptor when considering the regulation of Aβ clearance as disclosed above. The invention also encompasses a therapeutic composition comprising a nucleotide sequence encoding a polypeptide having an analogous function to the function of the IGF-I receptor.

Such a therapeutic composition can comprise a polynucleotide coding for an active fragment of the IGF-1 receptor as described above. In a particular embodiment, it comprises the pHIV-IGF1 R vector.

Process and Other Use of the Invention

According to another embodiment, the present invention provides a process for screening an active molecule interacting with the IGF-I receptor comprises administering said molecule to an animal during a time and in an amount sufficient for Alzheimer's disease-like disturbances to be modulated, wherein reversion of Alzheimer's disease-like disturbances is indicative of a molecule that increases IGF-I receptor activity and wherein appearance of Alzheimer's disease-like disturbances is indicative of a molecule that decreases IGF-I receptor activity. Advantegously, reversion of Alzheimer's disease-like disturbances is observed in an animal as defined above. The present invention will be more readily understood by referring to the following example. This example is illustrative of the wide range of applicability of the present invention and is not intended to limit its scope. Modifications and variations can be made therein without departing from the spirit and scope of the invention. Although any methods and materials similar or equivalent to those described herein can be used in the practice for testing of the present invention, the preferred methods and materials are described.

The following examples serve to illustrate the invention and must not be considered in a sense of limiting the scope thereof.

### EXAMPLE 1

### The Creation of a Viral Vector for Sustained Transgenic Expression

A viral vector was created as a genetic medium to introduce the mutated IGF-I receptor, referred to as IGF-IR.KR, in epithelial cells in the choroid plexus. IGF-IR.KR, the mutated IGF-I receptor, displays a K1003R mutation, where the lysine residue was substituted for an arginine residue, and acts as a dominant negative in recombination with the normal endogenous receptor, thereby disallowing normal function (Kato H, Faria T N, Stannard B, Roberts C T, Jr., LeRoith D (1993) Role of tyrosine kinase activity in signal transduction by the insulin-like growth factor-I (IGF-I) receptor. Characterization of kinase deficient IGF-I receptors and the main action of an IGF-I mimetic antibody (alpha IR-3). J Biol Chem 268: 2655-2661).

A lentiviral vector with prolonged expression characteristics was used (Consiglio A, Quattrini A, Martino S, Bensadoun J C, Dolcetta D, Trojani A, Bengalia G, Marchesini S, Cestari V, Oliverio A, Bordignon C, Naldini L (2001) In vivo gene therapy of metachromatic luekodystrophy by lentiviral vectors: correction of neuropathy and protection against learning impairments in affected mice. Nat Med 7: 310-316; Kordower J H, Emborg M E, Bloch J, Ma S Y, Chu Y, Levanthal L, McBride J, Chen E Y, Palfi S, Roitberg B Z, Brown W D, Holden J E, Pyzalski R, Taylor M D, Carvey P, Ling Z, Trono D, Hantraye P, Deglon N, Aebischer P, (2000) Neurodegeneration prevented by lentiviral vector delivery of GDNF in primate models of Parkinson's disease. Science 290: 767-773), derived from the human immunodeficiency type 1 virus (HIV-1), using vesicular stomatitis viruses (VSV) produced by transitory transfection and packaged in 293T cells plasmid vectors, following the concentration of said viral particles using ultra centrifugation, for the viral delivery. A third generation of HIV virus has been created following previously published methods (Dull H B (1998) Behind the AIDS mailer. Am J Prev Med 4: 239-240). For this process four constructions similar to those previously described were employed (Bosch A, Perret E, Desmaris N, Trono D, Heard J M. Reversal of pathology in the entire brain of mucopolysaccharidosis type VII mice after lentivirus-mediated gene transfer. Hum Gen Ther 8: 1139-1150, 2000):

(i) the RSV- Rev non over-lapping vector, which can read the nucleotide sequence which codifies the Rev protein for Roux sarcoma virus (RSV);
(ii) A p-RRE a conditionally cased vector which can read the nucleotide sequence which codifies the Rev response element (RRE);
(iii) A p-VSV vector which can read the nucleotide sequence which codifies the vector's heterogeneous packaging, especially the viral casing for vesicular stomatitis virus (VSV); and
(iv) A transfer vector bearing the genetic construction for the relevant transgene, which in this case is IGF-IR.KR, and the phosphoglycerolkinase (PCK) prompter which permits the transcription vector to be produced efficiently in the packaging cells (293T).

The first three vectors [1)-3)] are known (please refer to the previously quoted references). The construction of the last vector was carried out by introducing a HincII-XbaI fragment of the IGF-I receptor's cDNA that codifies the IGF-I receptor's mutated form, which in this case is the mutated receptor referred to as IGF-IR.KR which contains the mutation K1003R where the lysine residue has been substituted by an arginine residue (Kato H, Faria T N, Stannard B, Roberts C T, Jr., LeRoith D (1993) Role of tyrosine kinase activity in signal transduction by the insulin-like growth factor-I (IGF-I) receptor. Characterization of kinase deficient IGF-I receptors and the action of an IGF-I mimetic antibody (alpha IR-3) J Biol Chem 268: 2655-2661), in the vector HIV-LacZ (Naldini L, Blomer U, Gallay P, Ory D, Mulligan R, Gage F H, Verma I M, Trono D (1996) In vivo gene delivery and stable transduction of non-dividing cells by a lentiviral vector. Science 272: 263-267). In concise terms, the cDNA that codifies the mutated form of IGF-I bearing the mutation K1003R (IGF-IR.KR) was introduced into HIV-lacZ via information exchange from lacZ using the cDNA that codifies IGF-IR.KR according to the previously described methodology (Desmaris N, Bosch A, Salaun C, Petit C, Prevost M C, Tordo N, Perrin P, Schwartz O, de Rocquigny H, Heard J M (2001) Production and neurotropism of lentivirus vectors pseudotyped with lyssavirus envelope glycoproteins. Mol Ther 4: 149-156). For this process the HIV-lacZ vector was cut with SmaI/XbaI to eliminate the lacZ cDNA and was then bound with IGF-IR.KR codifying cDNA which was cut with HincII/XbaI. The restriction sites are homologous. As a result the transfer vector which bears the transgene IGF-IR.KR was obtained.

The lentiviral vector known as HIV/IGF-IR.KR or HIV/KR in this description was obtained through the transitory transfection of 293T cells. The RSV-Rev, the p-RRE, the p-VSV plasmids and the transfer vector bearing the transgene IGF-IR.KR are episomally packaged in the previously mentioned 293T cells (Desmaris N, Bosch A, Salaun C, Petit C, Prevost M C, Tordo N, Perrin P, Schwartz O, de Rocquigny H, Heard J M (2001) Production and neurotropism of lentivirus vectors pseudotyped with lyssavirus envelope glycoproteins. Mol Ther 4: 149-156). The 293T cellular line (commercially obtainable through the American Type Culture Collection) is a line of transformed epithelial human kidney cells that express the T antigen of SV40, which permits the episomal replication of the plasmids in the prompter region. On previous occasions the cells were planted in 10 cm plaques at a density of 1-5×10⁶ 24 hours before the transfection in a DMEM environment with 10% of foetal serum and and penicillin (100 IU/ml). During the transfection process a total of 32.75 μg of plasmid DNA per plate was used: 3 μg of p-VSV plasmids, 3.75 μg of RSV-Rev plasmids and 13 μg of both p-RRE plasmids and the transfer plasmid bearing the IGF-IR.KR transgene. The precipitate was obtained by adding 500 μl of HEPES 2× saline buffer solution (NaCl 280 mM, HEPES 100 mM, Na₂HPO₄ 1.5 mM, pH 7.12) drop by drop. While being shaken the precipitate was added to each cultivation tray. 10 ml of the medium was changed after 24 hours and after a further 24 hours the particles were collected and cleaned using a low speed centrifuge and passed through cellulose acetate filters (0.22 μm). Finally, following a series of ultra centrifuge processes the particles or lentiviral vectors HIV/IGF-IR.KR (HIV/KR), were re-suspended in a saline phosphate buffer (PBS/BSA) for later use. In concise terms, firstly the cultivation medium from the trays with the 293T cells was filtered using a 0.45 μm filter. This medium was then centrifuged at 4° C. for 1.5 hours at 19,000 rpm. The precipitate was re-suspended in 1% PBS/PBA and was left for 1 hour in ice and was then re-centrifuged for 1.5 hours at 19,000 rpm. The medium was then re-suspended in 1% PBS/BSA and then left in ice for 1 hour and centrifuged at 4° C. for 5 minutes at 14,000 rpm. The final product was immediately frozen and stored at −80° C. This same method was used to purify the empty HIV particles and the HIV/GFP particles. The empty HIV particles (or the empty HIV vectors), that correspond to the HIV-lacZ cut using SmaI/XbaI and the HIV/GFP particles have been described previously (Desmaris N, Bosch A, Salaun C, Petit C, Prevost M C, Tordo N, Perrin P, Schwartz O, de Rocquigny H, Heard J M (2001) Production and neurotropism of lentivirus vectors pseudotyped with lyssavirus envelope glycoproteins. Mol Ther 4: 149-156).

### EXAMPLE 2

### Lentiviral Vector Expression in Epithelial Cells from the Choroid Plexus

In order to analyse the expression of lentiviral vectors in epithelial cells from the choroid plexus a HIV/GFP lentiviral vector that contained the gene which codifies GFP as a transgene was constructed. In concise terms, the cDNA for the GFP protein gene was sub-cloned in a HIV-1 transfer vector [(pHR′CMV)-PGK in Desmaris N, Bosch A, Salaun C, Petit C, Prevost M C, Tordo N, Perrin P, Schwartz O, de Rocquigny H, Heard J M (2001) Production and neurotropism of lentivirus vectors pseudotyped with lyssavirus envelope glycoproteins. Mol Ther 4: 149-156], in BamHI/SalI restriction sites following on from the detailed description from Example 1, where the lentiviral vector referred to as HIV/GFP was obtained.

Following this, the animals, 5-6 month old male rats (n=7), were subjected to an injection using stereotaxical surgery using a Hamilton syringe, under tribromoethanol anaesthetic, containing 6 μl of HIV/GFP vector in both side ventricles (stereotaxical coordinates: 1 mm from the bregma, 1.2 mm to the side and 4 mm deep), at 1 μl per minute. Six months later the rats were sacrificed and the presence of the transgene was observed using fluorescence. For this purpose the animal was transcardially perfused with 4% paraformaldehyde. Then the brain was vibratome cut in 50 μm sections, and the sections were immediately mounted on gelatinized holders and the fluorescence of the GFP protein was directly observed using a fluorescence microscope (Leica).

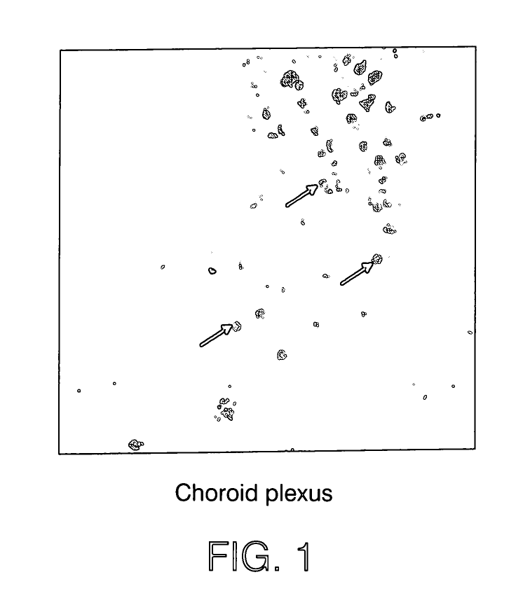

As a result it was determined that by administering the HIV/GFP lentiviral vector (the vector used in the invention of the codifying gene for the fluorescent GFP protein used as a transgene) to adult rats via intracerebroventricular (icv) injections results in the sustained expression of the GFP protein in the choroid plexus (FIG. 1).

### EXAMPLE 3

### The Transformation of Epithelial Cells from the Choroid Plexus Using the Lentiviral Vector HIV/IGF-IR.KR (HIV/KR)

The single layer of epithelial cells was obtained using a previously described method (Strazielle, N. and Ghersi-Egea, J. F. (1999) Demonstration of a coupled metabolism-efflux process at the choroid plexus as a mechanism of brain protection toward xenobiotics. J. Neurosci. 19: 6275-6289). 5-7 day old rats were sacrificed and the choroid plexus from the side and fourth ventricles were rapidly extracted and set in a DMEM cultivation medium on ice. Following their extraction and preparation the plexuses were digested using enzymes; 1 mg/ml of pronase (SIGMA) and 12.5 μg/ml Dnase I (Boehringer Mannheim), using simultaneous mechanical dispersion over a 15 minute period. Finally the solution was centrifuged (1,000 rpm) and the cells were re-suspended in DMEM with a 10% foetal serum (FCS) supplement, 10 ng/ml of EGF (Epidermal Growth Factor) (Sigma), 5 ng/ml of FGF (Fibroblast Growth Factor) (Boehringer Mannheim) and gentamicin. These cells were transformed with the lentiviral vector HIV/IGF-IR.KV (HIV/KR) and the empty HIV vector, using the following summarised method. After 24 hours of cultivation the medium was changed with fresh DMEM containing the virus (at least 50 μg/ml diluted at between 10⁻² and 10⁻³) and 8 μg/ml of polybrene (Sigma). This infective medium was replaced after 24 hours and the cells were maintained for another day and finally following suction of the medium the cells were processed.

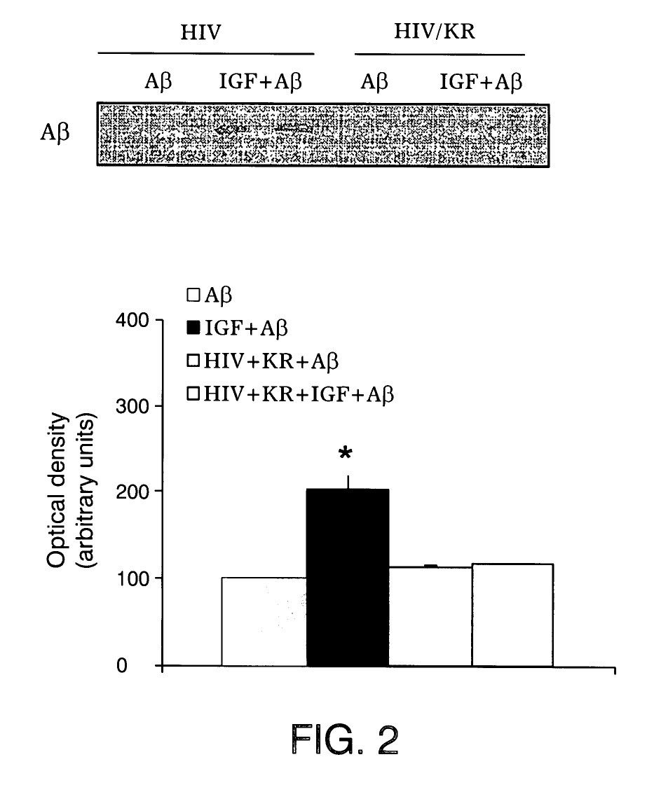

As a result the addition of the lentiviral vector HIV/IGF-IR:K (HIV/KR) to epithelial cells in cultivations obtained from rat choroid plexus was observed to produced a lower rate of the trophic factor IGF-I. Only in the cells infected with the HIV/KR vector, not those transfected with the empty HIV vector did the IGF-I fail to produce peptide transcytosis Aβ1-40 (FIG. 2). The transcytosis was quantified according to the amount of Aβ1-40 which passed from the upper cultivation chamber to the lower cultivation chamber, required the crossing of a single layer of epithelial cells (Carro E, Trejo J L, Gomez-Isla T, LeRoith D, Torres-Aleman I (2002) Serum insulin-like growth factor I regulates brain amyloid-beta levels. Nat Med 8: 1390-1937).

### EXAMPLE 4

### The Development of an Alzheimer Type Neuropathology in Healthy Adult Rats

Healthy adult rats were infected with Wistar strain using the HIV/IGF-IR.KR (HIV/KR) vector. This process was carried out using stereotaxical Surgery with a Hamilton syringe under tribromoethanol anaesthetic, containing 6 μl of HIV/IGF-IR.KR vector in both side ventricles (stereotaxical coordinates: 1 mm from the bregma, 1.2 mm to the side and 4 mm deep), at 1 μl per minute on 5-6 month old male rats. The control animals were injected with the same quantity of empty HIV viral vector under the same conditions. 5 months later the rats' cognitive capacity was measured using the Morris spatial learning test which relies on the hippocampus, a structure typically affected in Alzheimer (Clark C M, Karlawish J H (203) Alzheimer disease: current concepts and emerging diagnostic and therapeutic strategies. Ann Intern Med 138: 400-410), following standardized methodology (Trejo J L, Torres Aleman I (2001) Circulating insulin-like growth factor I mediates exercise-induced increases in the number of new neurons in the adult hippocampus. J Nuerosci 21: 1628-1634). This test known as the “water maze” (or the Morris test) determines spatial memory (van der Staay F J (2002) Assessment of age associated cognitive deficits in rats: a tricky business. Nuerosci Biobehav Rev 26: 753-759), which is one of the characteristic deficits presented in Alzheimer disease. On completion of the test the rats were sacrificed (6 months after being injected with the viral vector) and perfused via the aorta artery with saline buffer and their brains were immediately extracted, one hemisphere was stored at −80° C. for later processing using “western blot” and the other hemisphere was immersed in 4% paraformaldyhde for 24 hours for an immunohistochemistry study.

The levels of cerebral amyloid (Aβ) and the levels of CSF, cerebrospinal fluid, were determined using western blot techniques, ELISA and using immunocytochemistry, following previously described methodology (Carro E, Trejo J L, Gomez-Isla T, LeRoith D, Torres-Aleman I (2002) Serum insulin-like growth factor I regulates brain amyloid-beta levels. Nat Med 8: 1390-1937) and the levels of tau hyperphosphorylate (HPF-tau) in the cortex were also measured using western blot and immunocytochemistry (Carro E, Trejo J L, Gomez-Isla T, LeRoith D, Torres-Aleman I (2002) Serum insulin-like growth factor I regulates brain amyloid-beta levels. Nat Med 8: 1390-1937). In addition, the presence of HPF-tau deposits and amyloid deposits was also recorded using immunocytochemistry techniques (Carro E, Trejo J L, Gomez-Isla T, LeRoith D, Torres-Aleman I (2002) Serum insulin-like growth factor I regulates brain amyloid-beta levels. Nat Med 8: 1390-1937).

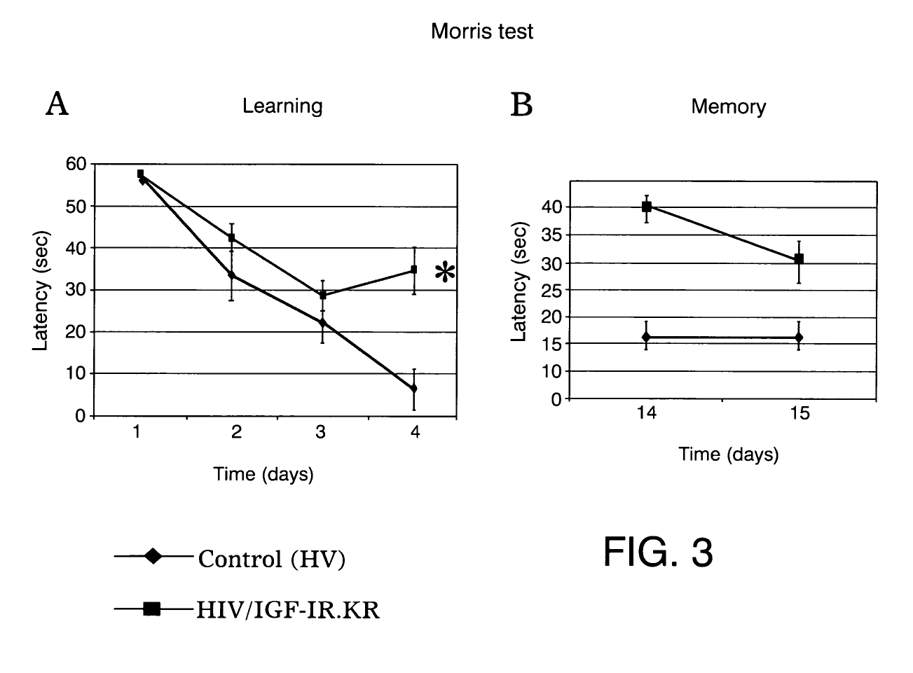

The animals with the blocked IGF-I signal within the choroid plexus due to the addition of the HIV/IGF-IR.KR vector showed significant cognitive deficits in spatial learning and memory (FIGS. 3A and 3B).

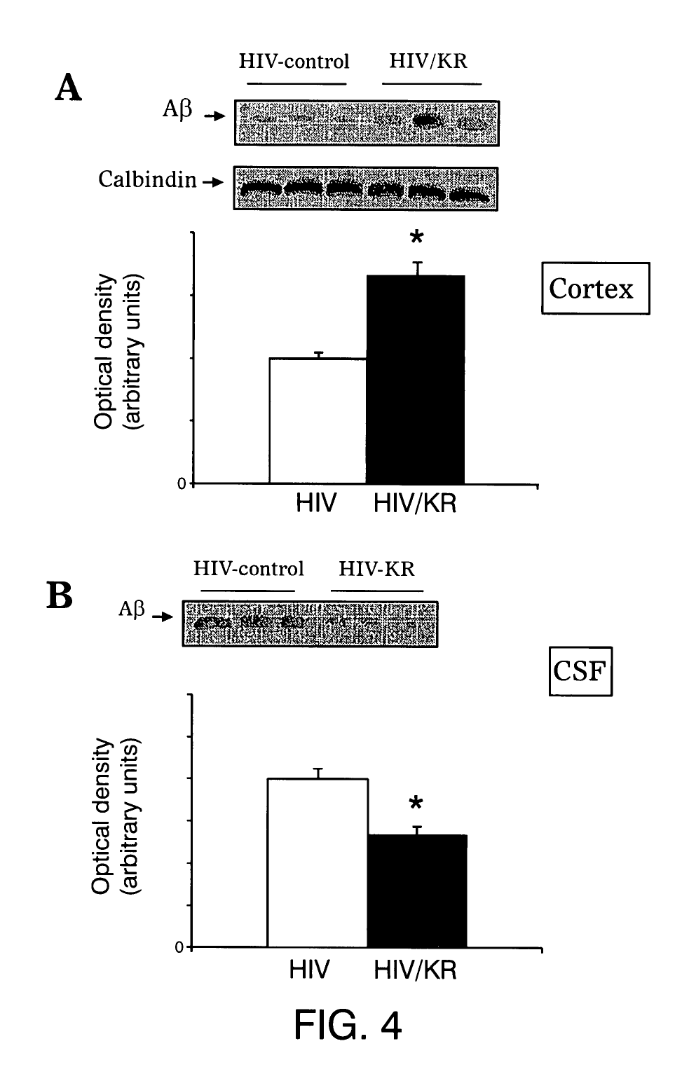

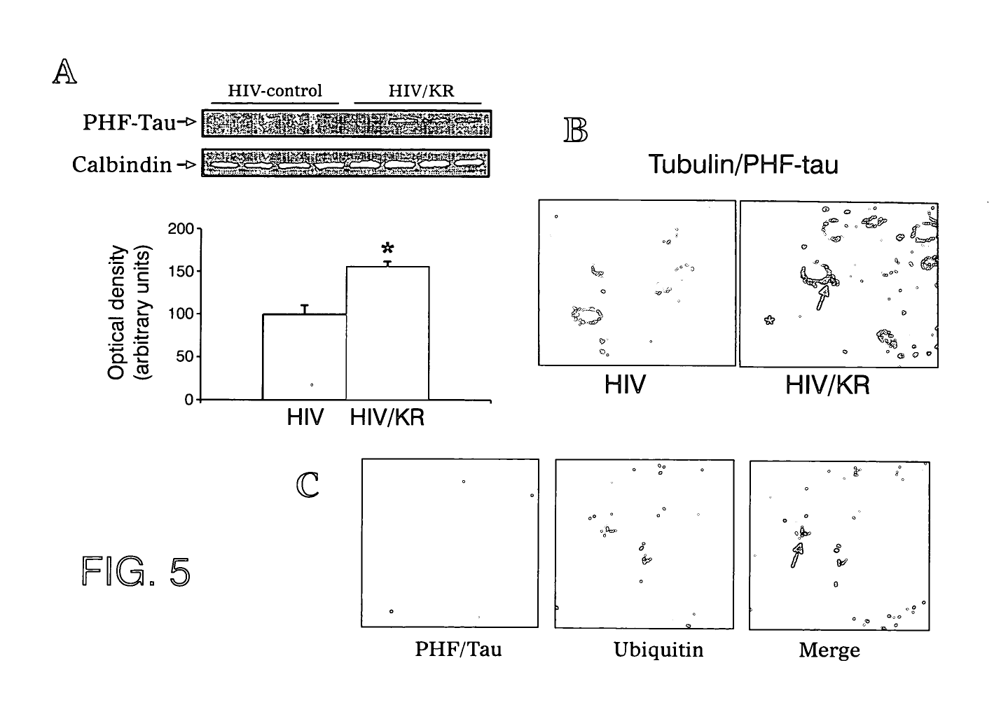

In addition a significant increase in the levels of Aβ was observed within the cerebral parachemistry compared to control animals and at the same time lower Aβ levels were observed in the CSF (FIGS. 4A and 4B). Both alterations are typical in Alzheimers disease (Selkoe D J (2001) Clearing the Brain's Amyloid Cobwebs. Neuron 32: 177-180; Sunderland T, Linker G, Mirza N, Putnam K T, Friedman D L, KImmel L H, Bergeson J, Manetti G J, Zimmermann M, Tang B, Bartko J J, Cohen R M (2003) Decreased beta-amyloid1-42 and increased tau levels in cerebrospinal fluid of patients with Alzheimer's disease. JAMA 289: 2094-2103). Along with this amyloidosis an intra and extra cellular HPF-tau accumulation was observed in telencephalic regions (FIG. 5). The extra cellular accumulations also contain ubiquitin (FIG. 5C) and are also characteristic in Alzheimer disease (Clark C M, Karlawish J H (2003) Alzheimer disease: current concepts and emerging diagnostic and therapeutic strategies. Ann Intern Med 138: 400-410). In addition, the animals showed Alzheimer type cellular alterations as they were seen to present reactive gliosis in association with the protein deposits and the significant synaptic protein deficits (Masliah E, Mallory M, Alford M, DeTeresa R, Hansen L A, McKeel D W, Jr., Morris J C (2001) Altered expression of synaptic proteins occurs early in the progression of Alzheimer disease. Neurology 56: 127-129). In conclusion, the animals injected with the prolonged expression lentiviral vector HIV/IGF-IR.KR (HIV/KR) presented neuropathological characteristics associated with Alzheimer's disease such as: high cerebral levels of amyloid, the presence of intra and extra cellular deposits of tau hyperphosphorylate and ubiquitin and cognitive deficiency.

### EXAMPLE 5

### The development of the Alzheimer Type Neuropathology in Genetically Modified Mice LID Mice

Another example of the experiment consisted in producing Alzheimer type pathological changes in transgenic mice. The chosen mice were old mice, to better simulate the normal conditions in which the Alzheimer pathology is developed in human beings.

The HIV/IGF-IR.KR (HIV/KR) vector was injected in 15 month old or older LID genetically modified transgenic mice. The transgenic mice used in this example are deficient in seric IGF-I following the elimination of the IGF-I hepatic gene using the Cre/Lox system (LID mice) (Yakar S, Liu J L, Stannard B, Butler A, Accili D, Sauer B, LeRoith D (1999) Normal growth and development in the absence of hepatic insulin-like growth factor I. Proc Natl Acad Sci USA 96: 7324-7329). LID mice already show some characteristics of Alzheimers per se, as the IGF-I deficit generates amyloidosis and gliosis (Carro E, Trejo J L, Gomez-Isla T, LeRoith D, Torres-Aleman I (2002) Serum insulin-like growth factor I regulates brain amyloid-beta levels. Nat Med 8: 1390-1937). In addition as the mice were old they showed cognitive deficiency and amyloidosis (Bronson R T, Lipman R D, Harrison D E (1993) Age-related gliosis in the white matter of mice. Brain Res 609: 124-128; van der Staay F J (2002) Assessment of age associated cognitive deficits in rats: a tricky business. Nuerosci Biobehav Rev 26: 753-759). The objective of this experiment was to obtain the most favourable conditions for amyloidosis production to determine if the system provided for this experiment generates amyloid deposits, one of the characteristics of Alzheimer's disease. The procedure and reactive material used are described in the previous examples. The animals were sacrificed three months after being injected with the viral vector.

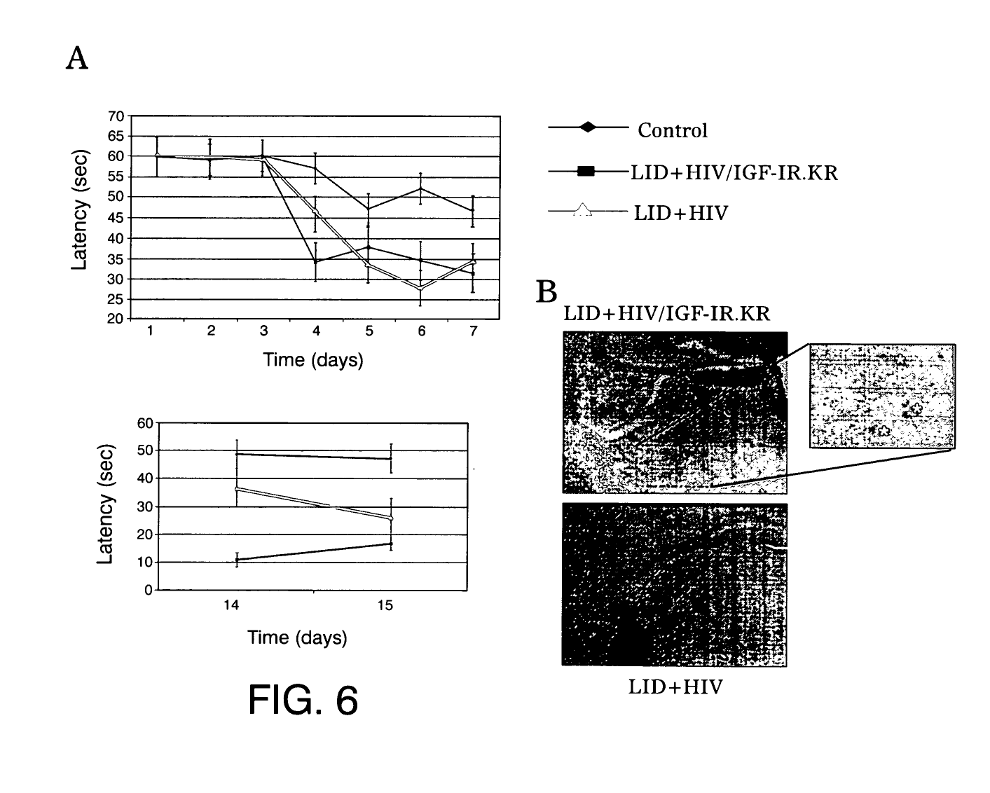

Just three months after the administration of the HIV/KR vector the old LID mice showed severe cognitive deficiency (FIG. 6A), and amyloidosis and taupathy similar to that observed in adult rats six months after being exposed to the viral vector (the results are similar to those described in FIGS. 4 and 5 although the data is not included). More importantly using this model a much more advanced state of the disease is achieved: the animals show amyloid accumulations, which although not congophilic (they are not detected with the insoluble plaque marker “Congo red”) they display typical diffused plaques (FIG. 6B).

### EXAMPLE 6

### Alzheimer's-Like Neuropathology After Blockade of Insulin-Like Growth Factor I Signaling in the Choroid Plexus

Aging, the major risk factor in Alzheimer's disease (AD)1 is associated to decreased input of insulin-like growth factor I (IGF-I), a purported modulator of brain β amyloid (Aβ) levels. The inventors now present evidence that reduced Aβ clearance due to impaired IGF-I receptor (IGF-IR) function originates not only amyloidosis but also other pathological traits of AD. Specific blockade of the IGF-IR in the choroid plexus, a brain structure involved in Aβ clearance by IGF-I, led to brain amyloidosis, cognitive impairment and hyperphosphorylated tau deposits together with other AD-related disturbances such as gliosis and synaptic protein loss. In old mutant mice with AD-like disturbances linked to abnormally low serum IGF-I levels, IGF-IR blockade in the choroid plexus exacerbated AD-like pathology. These findings shed light into the causes of late-onset Alzheimer's disease suggesting that an abnormal age-associated decline in IGF-I input to the choroid plexus contributes to development of AD in genetically-prone subjects.

Methods

Viral Vectors

Dominant negative (DN) and wild type (wt) IGF-I receptor (IGF-IR) cDNAs were subcloned in the Saml/Xbal site of the HIV-l-phosphoglycerate kinase 1 (PGK) transfer vector⁴⁰. The green fluorescent protein (GFP) cDNA was subcloned in the BamHI/Sall site. The HIV-I-PGK vector bound up in the Saml/Xbal site was used as a control (void vector). The packaging construct and the vesicular stomatitis virus G protein envelope included the pCMVΔR-8.92, pRSV-Rev and pMD.G plasmids⁴¹, respectively. The transfer vector (13 μg), the envelope (3.75 μg), and the packaging plasmids (3.5 μg) were co-transfected with calcium phosphate in 293 T cells (5×10⁶ cells/dish) cultured in Dulbecco's modified Eagle's medium (DMEM, Gibco, USA) with 10% FCS, 1% glutamine and 1% penicillin/streptomycin. Medium was changed 2 hrs prior to transfection and replaced after 24 hrs. Conditioned medium was collected 24 hrs later, cleared (1000 rpm/5 min), and concentrated ≈100 fold (19000 rpm/1.5 hrs). The pellet was re-suspended in phosphate-buffered saline with 1% bovine serum albumin, and the virus stored at −80<0>C. Viral title was determined by HIV-1 p24 ELISA (Perkin Elmer, USA).

Experimental Design

Wistar rats (5-6 months old, ˜300 g), and liver-IGF-l-deficient (LID) mice (6-21 months old, ˜25-30 g) were from our inbred colony. Animals were used following EEC guidelines. To minimize animal use the inventors initially compared responses of intact (sham) animals with those obtained in void-vector treated animals (see below) and since no differences were appreciated (see for example FIGS. 7d-f) the inventors used only the latter group as controls. Viral suspensions (140 μg HIV-1 p24 protein/ml, 6 μl/rat and 2 μl/mouse) were stereotaxically injected in each lateral ventricle (rat brain coordinates: 1 posterior from bregma, 1.2 lateral and 4 mm ventral; mouse: 0.6 posterior, 1.1 lateral and 2 mm ventral) with a 10 μl syringe at 1 μl/min. Recombinant IGF-I (GroPep, Australia) was labelled with digoxigenin (DIG, Pierce, USA) as described⁸ and administered as a bolus injection either into the brain parenchyma (1 μg/rat; stereotaxic coordinates: 3.8 posterior from bregma, 2 lateral and 3.2 mm ventral,) or through the carotid artery (10 μg/rat). Cerebrospinal fluid (CSF) was collected under anesthesia from the cisterna magna. Animals were perfused transcardially with saline buffer or 4% paraformaldehyde in 0.1 M phosphate buffer (PB, pH 7.4) for biochemical and immunohistochemical analysis, respectively. In in vitro studies a double-chamber choroid plexus epithelial cell culture system mimicking the blood-cerebrospinal (CSF) interface was used as described⁴. For viral infection, fresh DMEM containing the virus (≈1 μg/ml) and 8 μg/ml polybrene (Sigma) was added and replaced after 24 hrs. Cells were incubated another 24 hrs and thereafter IGF-I (100 nM) and/or DIG-albumin (1 μg/ml) added to the upper chamber. Lower chamber medium was collected and cells lysed and processed.

Immunoassays

Western-blot (WB) and immunoprecipitation were performed as described⁴². To analyze Aβ deposits, coronal brain sections were serially cut and pre-incubated in 88% formic acid and immunostained, as described⁴. For detection of total Aβ by ELISA, the inventors used the 4G8 antibody (Sigma) in the lower layer and anti-Aβ₁₋₄₀ or anti-Aβ₁₋₄₂ (Calbiochem, USA) in the top layer. To quantify both soluble and insoluble forms of Aβ, samples were extracted with formic acid and assayed as described⁴³. Human AD brain sections were obtained from Novagen (USA) and APP/PS2 mouse brain was a kind gift of H. Loetscher (Hoffman-La Roche, Switzerland). Mouse anti-Aβ (MBL, Japan) that recognizes rodent and human N-terminal Aβ forms, anti-albumin (Bethyl, USA), anti-transthyretin (Santa Cruz, USA), anti-apolipoprotein J (Chemicon, USA), anti-synaptophysin (Sigma), anti-dynamin 1 (Santa Cruz), anti-GFAP (Sigma), anti-calbindin (Swant, Switzerland), anti-β111-tubulin (Promega, USA), anti-PHF-tau (AT8, Innogenetics, Belgium), anti-ubiquitin (Santa Cruz), anti-pSer<9> and anti-pTyr<216> GSK3β (New England Biolabs, USA), anti-pAkt (Cell Signalling, USA) were all used at 1:500-1:1000 dilution. Secondary antibodies were Alexa-coupled (Molecular Probes, USA) or biotinylated (Jackson Immunoresearch, USA).

Behavioral Evaluation

Spatial memory was evaluated with the water maze test⁴⁴ as described in detail elsewhere⁴⁵. Briefly, after a 1 day habituation trial (day 1) in which preferences between tank quadrants were ruled out, for the subsequent 2-5/6 days the animals learned to find a hidden platform (acquisition), followed by one day of probe trial without the platform -in which swimming speed was found to be similar in all groups, and the preference for the platform quadrant evaluated. Nine to ten days later, animals were tested for long-term retention (memory) with the platform placed in the original location. On the last day, a cued version protocol was conducted to rule out possible sensorimotor and motivational differences between experimental groups. Behavioral data were analyzed by ANOVA and Student's t test.

Results

Blockade of IGF-I Signaling in the Choroid Plexus

Expression of a dominant negative (DN) form of the IGF-I receptor impairs IGF-I signaling⁷. Indeed, viral-driven expression of a DN IGF-IR (KR) in choroid plexus epithelial cells abolishes IGF-l-induced phosphorylation of its receptor and its downstream kinase Akt (FIG. 7a). The inventors previously found that IGF-I promotes the entrance of albumin through the choroid plexus into the CSF⁴. When choroid plexus cells are infected with the HIV-KR vector, IGF-I-induced transcytosis of albumin across the epithelial monolayer is inhibited (FIG. 7b). This indicates that blockade of IGF-IR function impairs passage of an Aβ carrier such as albumin through choroid plexus cells. Therefore, the inventors inhibited IGF-I signaling in the choroid plexus in vivo by intraventricular injection of the HIV-KR vector.

Delivery of HIV-GFP into the brain lateral ventricles (icv) resulted in sustained GFP expression in the choroid plexus epithelium of the lateral ventricles and adjacent periventricular cell lining (FIG. 7c). Vessels close to the injection site and the IV ventricle were also labelled (not shown). Using the same icv route, injection of the HIV-KR vector to rats resulted in blockade of IGF-IR function specifically in the choroid plexus, but not in brain parenchyma (FIG. 7d-f). Systemic injection of IGF-I in void vector- or saline-injected rats induces Akt phosphorylation in choroid plexus (FIG. 7d,e). Similarly, injection of IGF-I directly into the brain stimulates Akt phosphorylation in the parenchyma surrounding the injection site (FIG. 7f). However, in KR-injected animals, IGF-I phosphorylates Akt only when injected into the brain (FIG. 17f) but not after intracarotid injection (FIG. 7e), indicating blockade of systemic IGF-I input to the choroid plexus. In addition, passage of blood-borne digoxigenin-labeled IGF-I into the CSF was interrupted, as negligible levels of labeled IGF-I were found in the CSF after intracarotid injection (FIG. 7g). This suggests that intact IGF-IR function at the choroid plexus is required for the translocation of circulating IGF-I into the brain⁸. Altogether these results indicate that viral delivery of a DN IGF-IR into the choroid plexus results in effective blockade of IGF-IR function in this brain structure.

Development of AD-Like Neuropathology After Blockade of IGF-IR Function in the Choroid Plexus.

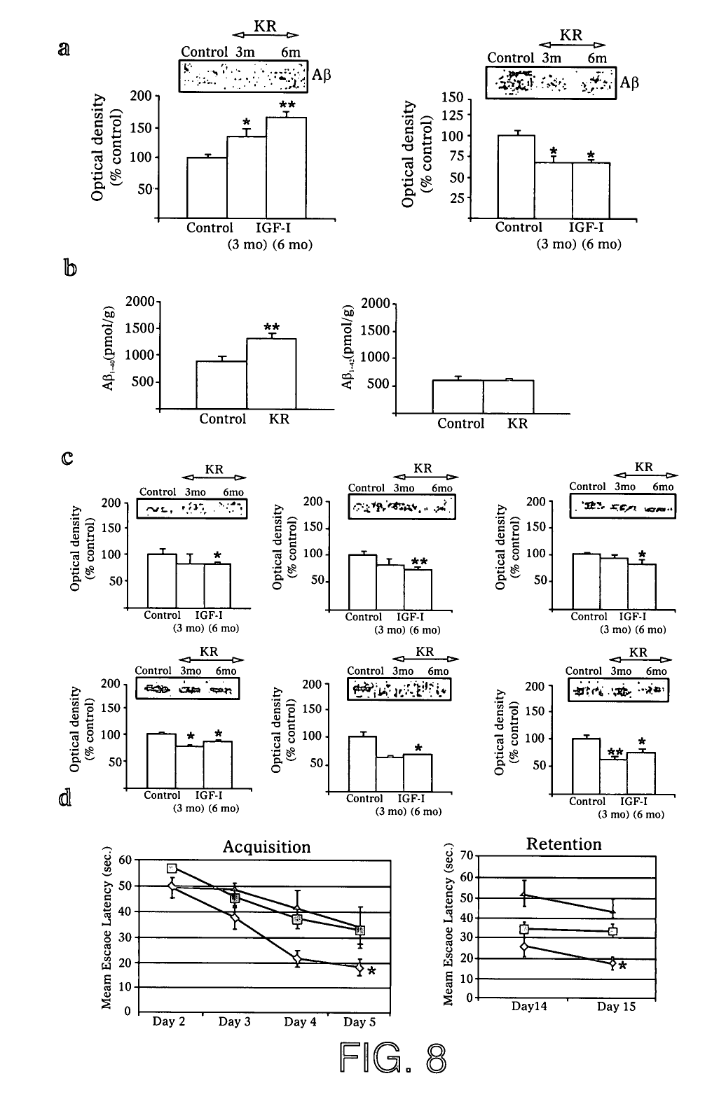

The inventors hypothesized that blockade of the IGF-IR in the choroid plexus would lead to increased brain Aβ due to reduced entrance of A[beta] carriers to the brain⁴. Indeed, after icv injection of HIV-KR, a progressive increase in Aβ₁₋ₓ levels in cortex (FIG. 8a) and hippocampus (not shown), but not in cerebellum (not shown) and a simultaneous decrease in Aβ₁₋ₓ levels in the CSF (FIG. 8a) was found using a pan-specific anti-Aβ. ELISA quantification of Aβ₁₋₄₀ and Aβ₁₋₄₂ showed increased βA₁₋₄₀ in cortex, while βA₁₋₄₂ remained unchanged six months after KR injection (FIG. 8b). No amyloid deposits were found in KR-injected rats using either Aβᵢ₋ₓ or Aβ₁₋₄₂-specific antibodies (not shown). A parallel decrease in brain and CSF levels of Aβ carriers such as albumin, apolipoprotein J and transthyretin was also found (FIG. 8c).

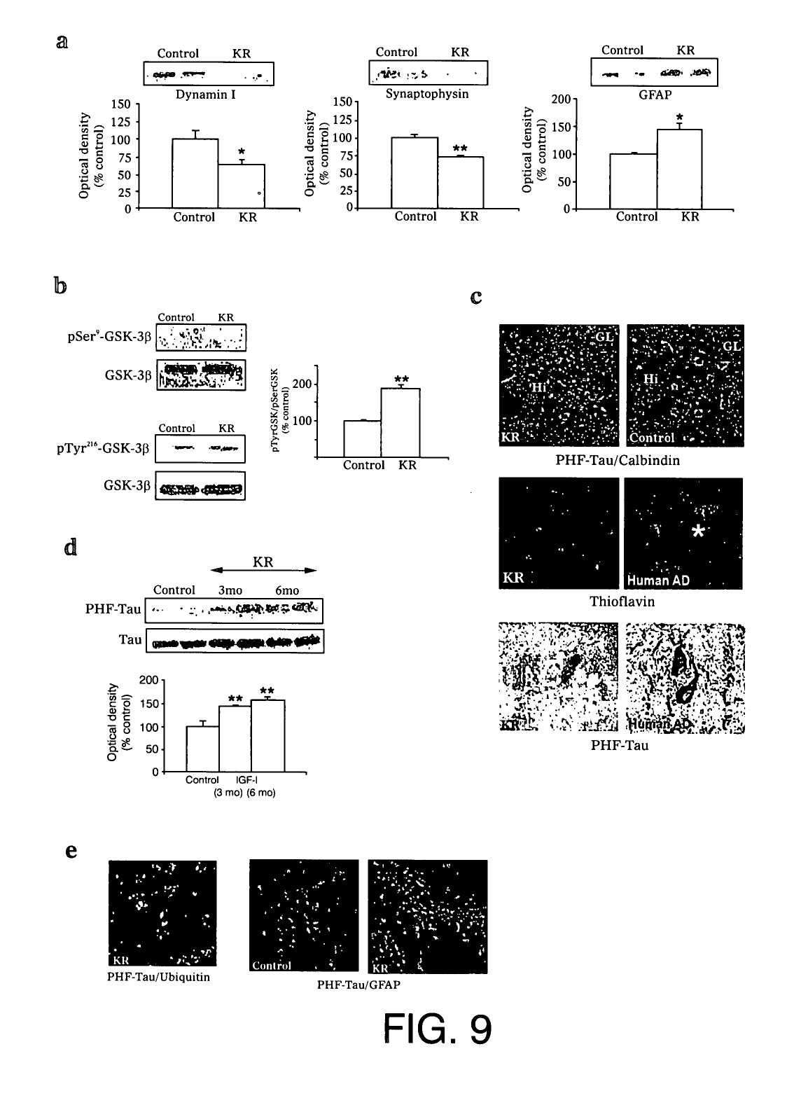

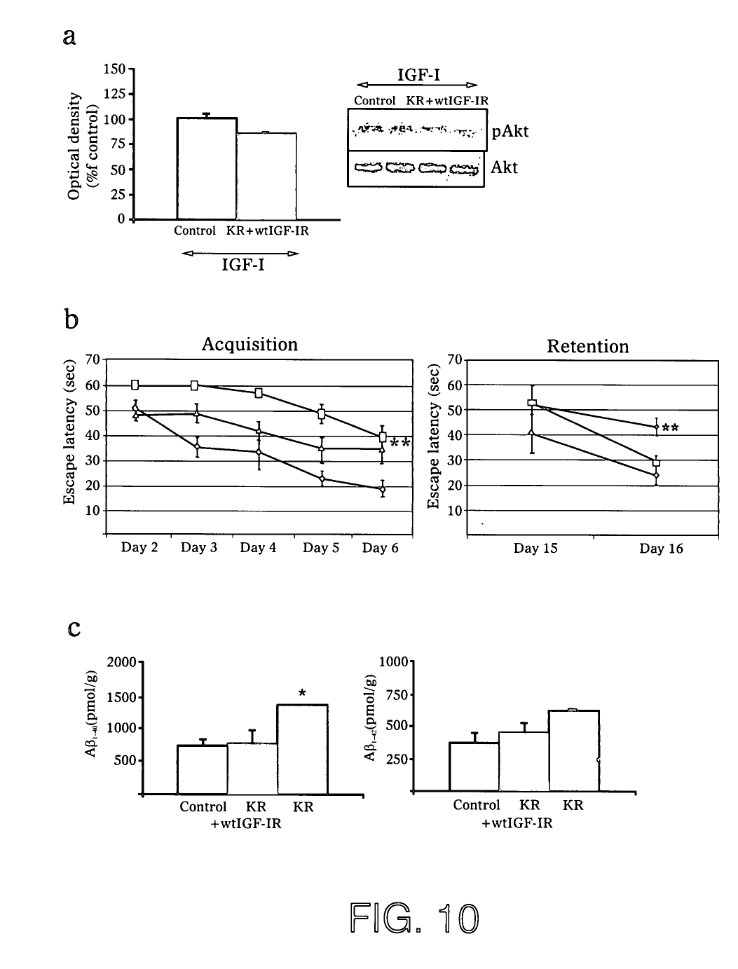

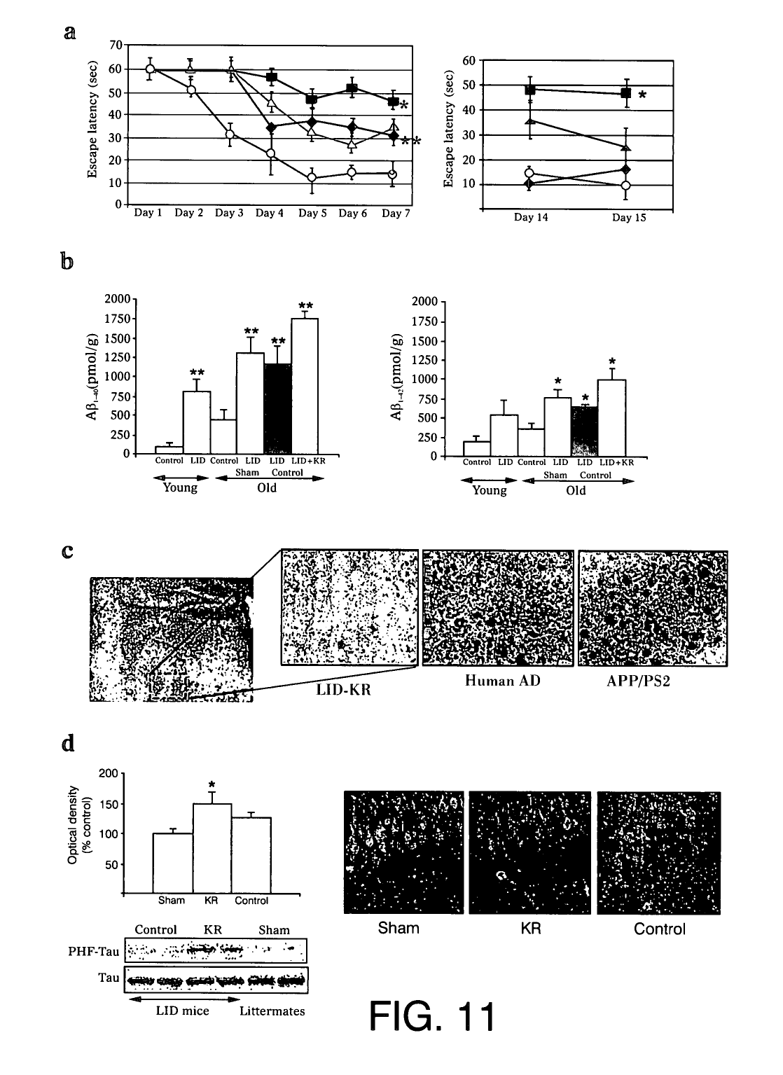

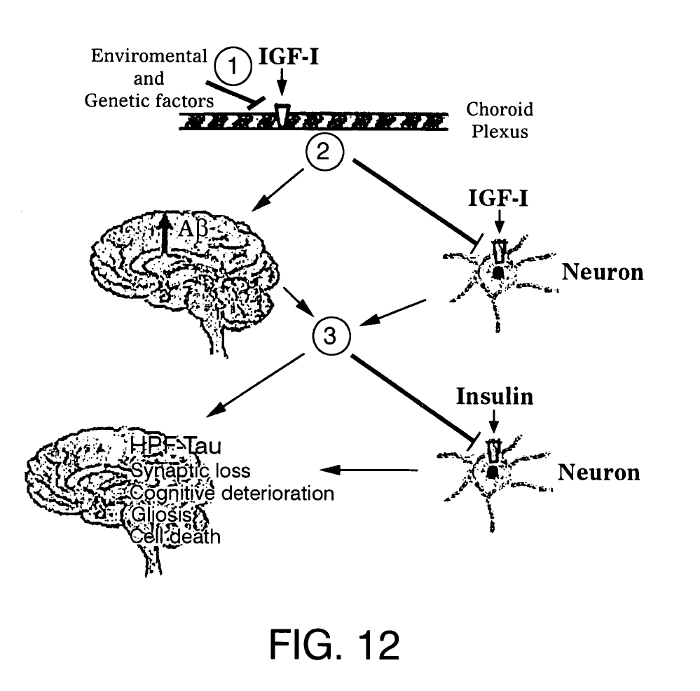

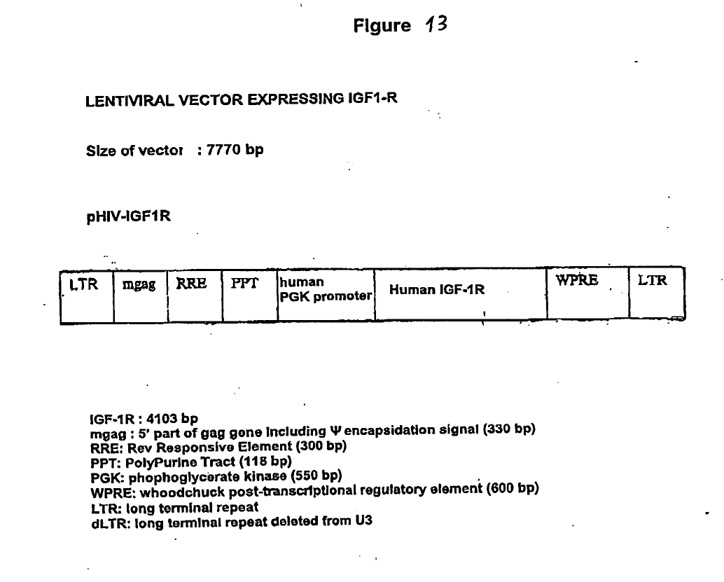

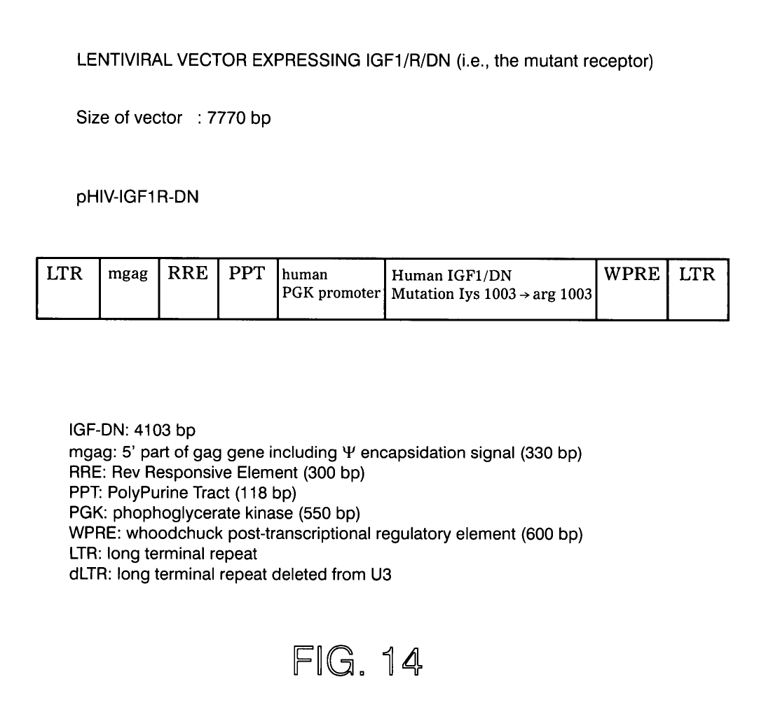

Since increased brain Aβ load, even in the absence of amyloid plaques, is associated to impaired cognition in animal models of AD⁹ the inventors determined whether KR-injected rats show learning and memory disturbances. Using the water maze test, an hippocampal-dependent learning paradigm widely used in rodent AD models¹⁰, the inventors found impaired performance in rats as early as 3 months after HIV-KR injection (FIG. 8d). Animals kept for 6 months after HIV-KR have similar cognitive perturbances (FIG. 8d). A decrease in the synaptic vesicle proteins synaptophysin and dynamin 1 is found in AD, a deficit that has been associated to cognitive loss^{11,12}. After KR injection both proteins are decreased (FIG. 9a) while GFAP, a cytoskeletal marker of gliosis associated to neuronal damage in AD¹¹, was elevated (FIGS. 9a,d).

Although amyloidosis is not always associated to the appearance of hyperphosphorylated tau (PHF-tau), the inventors found that 3 months after KR injection, when the animals have amyloidosis, they also have increased levels of PHF-tau. In addition, an increased pTyr²¹⁶GSK-3β (active form)/pSer⁹ GSK-3β (inactive form) ratio in the brain of KR-injected rats (FIG. 9b) suggested increased activity of this tau-kinase13, which agrees with appearance of intracellular deposits of PHF-tau in neurons (FIG. 9c) and glial cells (FIG. 9d, right panels). Using the AT8 antibody that recognizes PHF-tau in both pre-tangles and tangles¹⁴, intracellular deposits of PHF-tau and increased PHF-tau levels were observed in KR-rats (FIG. 9c). Comparison of KR rats with human AD suggested that intracellular PHF-tau deposits in the former correspond mostly to pre-tangles. Thus, thioflavin-S⁺ and PHF-tau⁺ tangle profiles were observed in human AD but not in KR rat brains (FIG. 9c, middle and lower left panels). PHF-tau deposits associated to ubiquitin and were surrounded by reactive glia (FIG. 9d). Robust PHF-tau staining was also observed in the choroid plexus of KR rats (not shown).

The inventors next restored IGF-IR function in the choroid plexus of rats injected with HIV-KR 3 months before by icv administration of HIV-wtlGF-IR. Animals were evaluated 3 months later to allow for IGF-IR functional recovery; i.e.: 6 months after the initial HIV-KR injection. Following restoration of IGF-IR signaling in the choroid plexus, as determined by normal levels of pAkt in the choroid plexus after intracarotid IGF-I (FIG. 10a), almost full recovery of brain function was achieved. Except for impaired learning (acquisition) in the water-maze (FIG. 10b) all other AD-like disturbances were reverted, including memory loss (FIG. 10, Table 1).

Blockade of IGF-IR Function in the Choroid Plexus Exacerbates AD-Like Traits in Old Mutant Mice.

Normal adult KR-treated rats do not develop plaques even though they have high brain Aβ¹⁻⁴⁰ levels. Absence of plaques may be because KR rats have unaltered levels of Aβ₁₋₄₂, the preferred plaque-forming Aβ peptide¹⁵ or because age-related changes in the brain may be necessary to develop plaques. However, it is well known that while aging rodents show a greater incidence of impaired cognition and increased brain Aβ levels, they do not develop Aβ plaques^{16,17}. Despite the latter, the inventors treated aged mutant LID mice¹⁸ with the KR vector. These mice have high brain levels of both Aβ₁₋₄₀ and Aβ₁₋₄₂ and show other age-related changes earlier in life, including low serum IGF-I and insulin resistance¹⁸ that may contribute to AD-like amyloidosis in the brain¹⁹. With this animal model the inventors aimed to better reproduce the conditions found in the aged human brain to gain further insight into the process underlying AD-like changes after blockade of choroid plexus IGF-IR.

Three months after KR injection, LID mice show disturbed water-maze learning and memory as compared to void-vector injected old LID mice (FIG. 11a). Significantly, aged control LIDs, as age-matched littermates, are already cognitively deteriorated when compared to young littermates (FIG. 11a). Therefore, blockade of IGF-IR function produces further cognitive loss. In addition, KR-injected old LID mice show increases in brain Aβ₁₋₄₀ and Aβ₁₋₄₂, as determined by ELISA but not significantly different from control old LID mice that had already high levels of both (FIG. 11b). LID-KR injected mice have small insoluble (formic-acid resistant) amyloid plaques that are also occasionaly found in old, but not young control LIDs (FIG. 11c). These deposits represent diffuse amyloid plaques²⁰ since they do not stain with Congo red or thioflavin-S as human AD plaques (not shown) and do not have the compact appearance of human AD or mutant mice amyloid plaques (FIG. 11c). Similarly to changes found in adult rats treated with the KR vector, old LID mice presented HPF-tau deposits and higher levels of HPF-tau 3 months after KR injection (FIG. 11d). Slightly higher GFAP levels (already significantly increased in control LID mice⁴), and synaptic protein loss were also found after KR injection in old LID mice (Table 2).

Discussion

These results indicate that IGF-IR blockade in the choroid plexus triggers AD-like disturbances in rodents including cognitive impairment, amyloidosis, hyperphosphorylated tau deposits, synaptic vesicle protein loss and gliosis. Most of these disturbances could be rescued by reverting IGF-IR blockade, although learning remained impaired. On the contrary, AD-like traits, in particular cognitive loss, were exacerbated when IGF-IR blockade was elicited in aged animals with lower than normal serum IGF-I levels. Although a general decrease in IGF-IR function is associated to normal aging²¹, these results suggest that loss of IGF-IR signaling in the choroid plexus may be linked to late-onset Alzheimer's disease²². While the causes of familial forms of AD-encompassing merely 5% of the cases¹, are slowly being unveiled, the etiology of sporadic AD is not established. Therefore, insight into mechanisms of reduced sensitivity to IGF-I at the choroid plexus may help unveil the origin of sporadic AD. For instance, risk factors associated to AD may contribute to a greater loss of IGF-IR function in the choroid plexus in affected individuals. Late-onset AD patients could present loss of sensitivity to the A[beta]reducing effects of IGF-I. Intriguingly, slightly elevated serum IGF-I levels were found in a pilot study of sporadic AD patients²³, a condition compatible with loss of sensitivity to IGF-I²⁴. Animal models of AD have successfully recreated several, but not all the major neuropathological changes of this human disease^{25,26}. Most have been developed through genetic manipulation of candidate disease-associated human proteins that usually include widespread expression of the mutated protein²⁷. Recently, a combined transgenic approach targeting three different AD-related proteins led to a mouse model that recapitulates the three main characteristics of AD: cognitive loss, amyloid plaques and tangles²⁸. In the present model, blockade of IGF-IR function specifically in the choroid plexus originates the majority of changes seen in AD brains except amyloid plaques and tangles. For instance, AD-like changes in our model include a reduction in dynamin 1 levels, also found in AD brains but not in animal models of AD amyloidosis¹², reduced CSF tranthyretin levels, also seen in AD²⁹, but not reported in animal models of the disease, or choroid plexus tauopathy, a common finding in AD patients³⁰. However, the lack of amyloid plaques and neurofibrillary tangles in the present model may question a significant pathogenic role of choroid plexus IGF-IR dysfunction in AD. It seems likely that additional factors, not reproduced in the present rodent model, are required to develop plaques and tangles. This is not surprising since under normal conditions rodents do not develop plaques or tangles³¹, unless forced to express mutant APP or tau (but see refs.^{32,33}). A shorter life-span, or structural differences in APP³¹ may account for this inter-species difference. In addition, while the largest amyloidosis the inventors observed was a mere ≈14-fold increase in total Aβ₁₋₄₀ after IGF-IR blockade in old LID mice, the aging human AD brain can produce substantial amounts of amyloid (well over 300-fold¹⁵), an effect that can be reproduced in rodent models of amyloidosis²⁷. Therefore, under proper experimental settings the rodent brain do produce plaques and tangles²⁸. Thus, the inventors hypothesize that the model recreates, within a rodent context, the initial stages of human sporadic Alzheimer's disease, when plaques and tangles are not yet formed.

Alternatively, development of plaques and tangles may be part of the pathological cascade idiosyncratic to humans (not reproducible in the normal rodent brain), and unrelated to the pathogenesis of the disease. As a matter of fact, the contribution of plaques and tangles to cognitive loss, the clinically relevant aspect of AD, is questionable. In agreement with the present findings, cognitive impairment may develop with brain amyloidosis without plaques³⁴. Similarly, high levels of HPF-tau without tangle formation are also associated to cognitive loss³⁵. Therefore, while current animal models of AD tend to emphasize the occurrence of plaques and tangles, the fact is that cognitive impairment does not depend in either one. Furthermore, amyloid plaques are not always associated to cognitive deterioration³⁶. At any rate, the present results reinforce the emerging notion that high amyloid and/or HPF-tau are sufficient to produce cognitive derangement.

The inventors previously found that serum IGF-I promotes brain Aβ clearance⁴. In response to blood-borne IGF-I, the choroid plexus epithelium translocates Aβ carrier proteins from the blood into the CSF. While low serum IGF-I levels, together with loss of sensitivity to IGF-I associated to aging³⁷ will affect target cells throughout the body, the inventors recently proposed that reduced IGF-I signaling specifically at the choroid plexus would interfere with Aβ clearance²². Indeed, the increase in brain Aβ together with decreased levels of Aβ carriers that we now found after IGF-IR blockade, support this notion. Notably, interruption of IGF-I signaling at the choroid plexus elicited not only amyloidosis but also other characteristic disturbances associated to AD. The amyloid hypothesis of AD favors accumulation of amyloid as the primary pathogenic event². However, the factors contributing to amyloid deposition in sporadic AD are not known. Both impaired degradation of Aβ and/or clearance, or excess production could be responsible. The present results indicate that Aβ accumulation due to impaired clearance may be sufficient to initiate the pathological cascade. In this sense, the primary disturbance would be loss of function of the IGF-IR at the choroid plexus, which in turn may originate the pathological cascade due to excess amyloid<2>. Therefore, by placing loss of IGF-I input upstream of amyloidosis the inventors can easily reconcile their observations with current pathogenic concepts of late-onset AD (FIG. 11).

Nevertheless, the inventors' observations leave open several issues. The inventors cannot yet determine the hierarchical relationship between tauopathy and amyloidosis because in their study accumulation of PHF-tau coincided in time with high levels of Aβ. In addition, the inventors observed increases in Aβ₁₋₄₀ but not in in KR-injected rats. This agrees with the observation that the greatest increase in human AD is in Aβ₁₋₄₀, but Aβ₁₋₄₂ also increases in humans³⁸. Since increases in Aβ₁₋₄₂ are found in mutant LID mice⁴, life-long exposure to low IGF-I input may be necessary for Aβ₁₋₄₂ to accumulate in rodent brain within a wild type background of APP and APP-processing proteins. Finally, while reversal of IGF-IR blockade in the choroid plexus rescued most AD-like changes, the animals still have deranged learning. Therefore, AD-like changes following IGF-IR blockade may compromise learning abilities even after been reverted, a finding that differs from that observed in current models of AD amyloidosis where reduction of amyloid load usually accompanies cognitive recovery³⁹.

In conclusion, by specifically blocking IGF-IR function in the choroid plexus (as opposed to the general loss of IGF-I input associated to aging³⁷) the inventors have unveiled a mechanism whereby pathognomonic signs of AD develop. This occurs within a wild type background of AD-relevant proteins such as APP or tau, resembling more closely sporadic forms of human AD. The non-human model of the present invention is relevant for analysis of pathogenic pathways in AD, definition of new therapeutic targets and drug testing. In this regard, blockade of IGF-IR in animal models of AD and AD-related pathways may help gain insight into the interactions between pathogenic routes, risk factors and secondary disturbances. Because the inventors' observations favor that late-onset AD is related to age-dependent reduction in Aβ clearance, drug development may be aimed towards its enhancement. Based on the success in developing insulin sensitizers for type 2 diabetes, enhancement of sensitivity to IGF-I in AD patients may be already within reach since the two hormones share common intracellular pathways.

TABLE 1
Restoring IGF-IR function in the choroid plexus of KR-injected
rats with HIV-wtIGF-1R reverts AD-like changes in brain
levels of various AD-related proteins

| | KR | KR+wt IGF-IR |
|---|---|---|
| AD-related proteins | (% Control) | (% Control) |
| Aβ₁₋ₓ | 179 ± 8* | 101 ± 30 |
| PHF-Tau | 154 ± 7** | 99 ± 5 |
| GFAP | 198 ± 29* | 119 ± 11 |
| Synaptophysin | 72 ± 1** | 108 ± 4 |
| Dynamin 1 | 64 ± 5* | 102 ± 5 |

Protein levels were determined by WB and quantified by densitometry. Control, void- vector injected rats, n = 7; KR, n = 7; KR+wtlGF-IR n = 7.
*p < 0.05 and
**p < 0.01 vs control.

TABLE 2
Blockade of IGF-IR in choroid plexus of serum
IGF-I deficient (LID) old mice results in AD-like
changes in various AD-related proteins.

| | LID-KR |
|---|---|
| AD-related proteins | (% Control) |
| GFAP | 112 ± 2* |
| Synaptophysin | 50 ± 2** |
| Dynamin 1 | 85 ± 1.5** |

Protein levels were determined by WB and quantified by densitometry. Control, void- vector injected old LID mice, n = 5; LID-KR, n = 5.
*p < 0.05 and
**p < 0.01 vs control.

### REFERENCE LIST

1. Mayeux, R. Epidemiology of neurodegeneration. Annu. Rev. Neurosci. 26, 81-104 (2003).
2. Selkoe. D. J. Clearing the Brain's Amyloid Cobwebs. Neuron 32, 177-180 (2001).
3. Golde, T. E. Alzheimer disease therapy: can the amyloid cascade be halted? J. CHn Invest W, 11-18 (2003).
4. Carro, E., Trejo. J. L, Gomez-lsla, T., LeRoith. D. & Torres-Aleman J. Serum insulin-like growth factor I regulates brain amyloid-beta levels. Nat. Med. 8, 1390-1397 (2002).
5. DeMattos. R. B. et al. Peripheral anti-A beta antibody alters CNS and plasma A beta clearance and decreases brain A beta burden in a mouse model of Alzheimer's disease. Proc. Natl. Acad. ScL U.S.A 98, 8850-8855 (2001).
6. Banks. W. A., Robinson, S. M., Verma. S. & Morley. J. E. Efflux of human and mouse amyloid beta proteins 1-40 and 1-42 from brain: impairment in a mouse model of Alzheimer's disease. Neuroscience 121, 487-492 (2003).
7. Fernandez, A. M. et al. Functional inactivation of the IGF-I and insulin receptors in skeletal muscle causes type 2 diabetes. Genes Dev. 15, 1926-1934 (2001).
8. Carro. E., Nunez[Lambda], Busiguina. S. & Torres-Aleman J. Circulating insulin-like growth factor I mediates effects of exercise on the brain. J. Neurosci. 20, 2926-2933 (2000).
9. Holcomb. L A. et al. Behavioral changes in transgenic mice expressing both amyloid precursor protein and presenilin-1 mutations: lack of association with amyloid deposits. Behav. Genet. 29, 177-185 (1999). 10. D'Hooge. R. & De Deyn. P. P. Applications of the Morris water maze in the study of learning and memory. Brain Res. Brain Res. Rev. 36, 60-90 (2001).
10. D'Hooge, F. & De Deyn, P. P. Applications of the Morris water maze in the study of learning and memory. Brain Res. Brain Res. Rev. 36, 60-90 (2001).
11. Ingelsson. M. et al. Early Abeta accumulation and progressive synaptic loss, gliosis, and tangle formation in AD brain. Neurology 62, 925-931 (2004).
12. Yao. P. J. et al. Defects in expression of genes related to synaptic vesicle trafficking in frontal cortex of Alzheimer's disease. Neurobiol. Dis. 12, 97-109(2003).
13. Liu, S. J. et al. Overactivation of glycogen synthase kinase-3 by inhibition of phosphoinositol-3 kinase and protein kinase C leads to hyperphosphorylation of tau and impairment of spatial memory. J. Neurochem. 87, 1333-1344 (2003).
14. Lauckner. J., Frey. P. & Geula. C. Comparative distribution of tau phosphorylated at Ser262 in pre-tangles and tangles. Neurobiol. Aging 24, 767-776 (2003).
15. Gravina. S. A. et al. Amyloid beta protein (A beta) in Alzheimer's disease brain. Biochemical and immunocytochemical analysis with antibodies specific for forms ending at A beta 40 or A beta 42(43). J. Biol. Chem. 270, 7013-7016 (1995).
16. van der Staay, F. J. Assessment of age-associated cognitive deficits in rats: a tricky business. Neurosci. Biobehav. Rev. 26, 753-759 (2002).
17. Vaucher, E. et al. Amyloid beta peptide levels and its effects on hippocampal acetylcholine release in aged, cognitively-impaired and -unimpaired rats. J. Chem. Neuroanat. 21, 323-329 (2001).
18. Yakar. S. et al. Normal growth and development in the absence of hepatic insulin-like growth factor I. Proc. Natl. Acad. Sci. U.S.A 96, 7324-7329 (1999).
19. Gasparini. L. & Xu1H. Potential roles of insulin and IGF-1 in Alzheimer's disease. Trends Neurosci. 26, 404-406 (2003).
20. Ikeda. S., Allsop. D. & Glenner. G. G. Morphology and distribution of plaque and related deposits in the brains of Alzheimer's disease and control cases. An immunohistochemical study using amyloid beta-protein antibody. Lab Invest 60, 113-122 (1989).
21. Lieberman. S. A., Mitchell, A. M., Marcus. R., Hintz, R. L. & Hoffman. A-R- The insulin-like growth factor I generation test: resistance to growth hormone with aging and estrogen replacement therapy. Horm. Metab Res. 26, 229-233 (1994).
22. Carro. E. & Torres-Aleman J. The role of insulin and insulin-like growth factor I in the molecular and cellular mechanisms underlying the pathology of Alzheimer's disease. Eur. J. Pharmacol. 490, 127-133 (2004).
23. Tham. A. et al. Insulin-like growth factors and insulin-like growth factor binding proteins in cerebrospinal fluid and serum of patients with dementia of the Alzheimer type. J. Neural Transm. Park Dis. Dement. Sect. 5, 165-176 (1993).
24. Jain, S., Golde. D. W., Bailey. R. & Geffner, M. E. Insulin-like growth factor-l resistance. Endocr. Rev. 19, 625-646 (1998).
25. Hock, B. J., Jr. & Lamb, B. T. Transgenic mouse models of Alzheimer's disease. Trends Genet. 17, S7-12 (2001). 26. Schwab, C, Hosokawa. M. & McGeer, P. L Transgenic mice overexpressing amyloid beta protein are an incomplete model of Alzheimer disease. Exp. Neurol. 188, 52-64 (2004).
27. Kawarabayashi. T. et al. Age-dependent changes in brain, CSF, and plasma amyloid (beta) protein in the Tg2576 transgenic mouse model of Alzheimer's disease. J. Neurosci. 21, 372-381 (2001).
28. Oddo. S. et al. Triple-transgenic model of Alzheimer's disease with plaques and tangles: intracellular Abeta and synaptic dysfunction. Neuron 39, 409-421 (2003).
29. Serot J. M., Bene, M. C. & Faure. G. C. Choroid plexus, ageing of the brain, and Alzheimer's disease. Front Biosci. 8, S515-S521 (2003).
30. Miklossy. J. et al. Curly fiber and tangle-like inclusions in the ependyma and choroid plexus-a pathogenetic relationship with the cortical Alzheimer-type changes? J. Neuropathol. Exp. Neurol. 57, 1202-1212 (1998).
31. De Strooper. B. et al. Production of intracellular amyloid-containing fragments in hippocampal neurons expressing human amyloid precursor protein and protection against amyloidogenesis by subtle amino acid substitutions in the rodent sequence. EMBO J. 14, 4932-4938 (1995).
32. Capsoni. S. et al. Alzheimer-like neurodegeneration in aged antinerve growth factor transgenic mice. Proc. Natl. Acad. ScL U.S.A 97, 6826-6831 (2000).
33. Little. C. S., Hammond, C J., Maclntyre. A-, Balin, B. J. & Appelt. D. M. Chlamydia pneumoniae induces Alzheimer-like amyloid plaques in brains of BALB/c mice. Neurobiol. Aging 25, 419-429 (2004).
34. Kumar-Singh, S. et al. Behavioral disturbances without amyloid deposits in mice overexpressing human amyloid precursor protein with Flemish (A692G) or Dutch (E693Q) mutation. Neurobiol. Dis. 7, 9-22 (2000).
35. Avila. J., Lucas, J. J., Perez. M. & Hemandez, F. Role of tau protein in both physiological and pathological conditions. Physiol Rev. 84, 361-384 (2004).
36. Savonenko. A. V., Xu. G. M., Price. D. L, Borchelt, D. R. & Markowska, A. L. Normal cognitive behavior in two distinct congenic lines of transgenic mice hyperexpressing mutant APP SWE. Neurobiol. Dis. 12, 194-211 (2003).
37. Arvat. E., Broglio, F. & Ghigo, E. Insulin-Like growth factor I: implications in aging. Drugs Aging 16, 29-40 (2000).
38. Wang, J., Dickson. D. W., Trojanowski. J. Q. & Lee. V. M. The levels of soluble versus insoluble brain Abeta distinguish Alzheimer's disease from normal and pathologic aging. Exp. Neurol. 158, 328-337 (1999).
39. Younkin. S. G. Amyloid beta vaccination: reduced plaques and improved cognition. Nat. Med. 7, 18-19 (2001).
40. Naldini, L. et al. In vivo gene delivery and stable transduction of nondividing cells by a lentiviral vector. Science 272, 263-267 (1996).
41. Desmaris. N. et al. Production and neurotropism of lentivirus vectors pseudotyped with lyssavirus envelope glycoproteins. MoI. Ther. 4, 149-156 (2001).
42. Pons, S. & Torres-Aleman, l. Insulin-like Growth Factor-I Stimulates Dephosphorylation of lkappa B through the Serine Phosphatase Calcineurin(Protein Phosphatase 2B). J. Biol. Chem. 275, 38620-38625 (2000).
43. Suzuki, N. et a/. An increased percentage of long amyloid beta protein secreted by familial amyloid beta protein precursor (beta APP717) mutants. Science 264, 1336-1340 (1994).
44. Morris, R. Developments of a water-maze procedure for studying spatial learning in the rat. J. Neurosci. Methods 11, 47-60 (1984).
45. Carro. E., Trejo. J. L, Busiguina, S. & Torres-Aleman. l. Circulating insulin-like growth factor I mediates the protective effects of physical exercise against brain insults of different etiology and anatomy. J. Neurosci. 21, 5678-5684 (2001).
46. Xie. L et al. Alzheimer's beta-amyloid peptides compete for insulin binding to the insulin receptor. J. Neurosci. 22, RC221 (2002).

1. A non-human animal used as a model for disease where abnormal brain accumulation of [beta] amyloid and/or amyloid plaques are involved, wherein [beta] amyloid clearance from brain is decreased.

2. A non-human animal model according to claim 1, wherein said animal displays an alteration in the biological activity of the receptor of the insulin type I-like growth factor (IGF-I) located in the epithelial cells of the choroids plexus from the cerebral ventricles.

3. An animal model according to claim 2, wherein said alteration of the biological activity of the IGF-I receptor consisting in biological elimination.

4. An animal model according to both claim 1 wherein said animal is a mammal.

5. An animal model according to claim 4, wherein said mammal is selected from rodents and primates.

6. An animal model according to claim 5, wherein said roden is a rat or a mouse.

7. An animal model according to claim 2, wherein said alteration in the IGF-I receptor functions in the epithelial cells located in the choroids plexus is due to the expression of a dominant non-functional mutated form of said IGF-I receptor.

8. The animal model according to claim 3, wherein said elimination of IGF-I receptor biological activity is achieved by a gene transfer vector derived from HIV or AAV.

9. The animal model according to claim 8, wherein said vector was deposited at CNCM on Nov. 10, 2004 under accession number I-3316.

10. An animal model according to claim 7, wherein the afore mentioned dominant non functional mutated form of the IGF-I receptor is the non functional mutated form of the IGF-I receptor referred to as IGF-IR.KR which displays the K1003R mutation, in which the lysine residue found in position 1003 in the IGF-I receptor amino acid sequence has been substituted by an arginine residue.

11. An animal which according to claim 7, wherein said dominant non functional mutated form of the IGF-I receptor is the mutated form of the IGF-I non functional receptor referred to as IGF-IR.KR which contains the K 1 003A mutation, in which the lysine residue in position 1003 of the receptor amino acids sequence for the human IGF-I has been substituted with an alanine residue.

12. An animal model according to claim 1, wherein said model is a normal animal.

13. An animal model according to claim 12 wherein said animal is a normal healthy rat.

14. An animal model according to claim 12 wherein said animal is transgenic.

15. An animal model according to claim 14 wherein said transgenic animal is a LID transgenic mouse.

16. An animal model according to claim 1, wherein said animal is useful as an experimental model for a neurodegenerative disease.

17. An animal model according to claim 16, where said neurodegenerative disease is Alzheimer's disease.

18. A procedure for obtaining of a non-human animal useful as an experimental model described in any of claims 1-1y, wherein said procedure includes the elimination of the functional activity of the IGF-I receptor in epithelial cells in the choroid plexus in said non-human animal using a transgenesis process.

19. A procedure according to claim 18, wherein said transgenesis includes the administration of epithelial cells from the choroids plexus of a non-human animal developed with a genetic make-up that includes a polynucleotide with a nucleotide sequence that encodes a dominant non-functional mutated form of the IGF-I receptor, or a vector that can read said genetic structure to enable the transformation of said epithelial cells from the choroid plexus in a way which expresses said dominant non-functional mutated form of the IGF-I receptor.

20. A procedure which according to claim 19, wherein the administration of said genetic construction or said vector to said epithelial cells from the choroid plexus will be carried out using a intracerebroventricular injection (icv).

21. A procedure which according to claim 19, wherein said vector is selected from viral and non-viral vectors.

22. A procedure which according to claim 21, which the viral vector is a lentiviral vector or an adeno-associated viral vector.

23. A procedure which according to claim 19, wherein said dominant non functional mutated form of the IGF-I receptor is the mutated form of the IGF-I non-functional receptor referred to as IGF-IR.KR which contains the K1003R mutation, in which the lysine residue in position 1003 of the IGF-I receptor amino acids sequence has been substituted with an arginine residue.

24. A procedure which according to claim 19, wherein said dominant non functional mutated form of the IGF-I receptor is the mutated form of the IGF-I non functional receptor referred to as IGF-IR.KR which contains the K1003A mutation, in which the lysine residue in position 1003 of the human IGF-I receptor amino acids sequence has been substituted with an alanine residue.

25. A procedure according to claim 19, wherein said animal is a normal non-human animal.

26. A procedure which according to claim 19, wherein said non-human animal is a non-human transgenic animal.

27. A procedure which according to claim 18, wherein said transgenesis process for the elimination of the functional activity of the IGF-I receptor includes the transformation of the epithelial cells from the choroids plexus of a non-human animal by introducing a genetic construction which can interpret a polynucleotide whose nucleotide sequence codifies an inhibition element on the expression of IGF-I receptor gene capable of eliminating it's biological activity, or a vector which includes said genetic construction, where the inhibitor element is selected from:
a) A sequence of antisense nucleotides specifies the gene sequence or the sequence for the IGF-I mRNA receptor;
b) A specific mRNA ribozyme from the IGF-I receptor;
c) A specific mRNA aptamer from the IGF-I receptor and;
d) A specific mRNA RNA interference (RNAi) from the IGF-I receptor.

28. The procedure according to claim 18, wherein said transgenesis process includes the administration of a genetic construction able to read the specific prompter for the choroid plexus and a polynucleotide whose sequence codifies the dominant non functional mutated form of the IGF-I receptor, or a vector that can read said genetic construction, from embryonic cells from the non-human animal.

29. A procedure where according to claim 28, wherein said dominant non functional mutated form of the IGF-I receptor is the mutated form of the IGF-I non functional receptor referred to as IGF-IR.KR which contains the K1003R mutation, in which the lysine residue in position 1003 of the IGF-I receptor amino acids sequence has been substituted with an arginine residue.

30. A procedure where according to claim 28, wherein said dominant non functional mutated form of the IGF-I receptor is the mutated form of the IGF-I non functional receptor referred to as IGF-IR.KR which contains the K1003A mutation, in which the lysine residue in position 1003 of the human IGF-I receptor amino acids sequence has been substituted with an alanine residue.

31. The procedure according to claim 18, wherein said transgenesis includes the administration of a genetic construction able to read the specific prompter for the choroid plexus and a polynucleotide whose sequence codifies the dominant non functional mutated form of the IGF-I receptor, or a vector that can read said genetic construction, from embryonic cells from the non human animal, where the inhibitor element is selected from:
a) a sequence of antisense nucleotides specifies the gene sequence or the sequence for the IGF-I mRNA receptor,
b) A specific mRNA ribozyme from the IGF-I receptor,
c) A specific mRNA aptamer from the IGF-I receptor and,
d) A specific mRNA RNA interference (RNAi) from the IGF-I receptor.

32. Procedure according to claim 28, wherein said prompter specific to the tissue is a transthyretin gene prompter.

33. Procedure according to claim 28, wherein said transgenesis process is non-deductible.

34. A gene transfer vector as defined in claim 8, wherein said vector is selected from a lentiviral vector and an adeno-associated vector.

35. A gene transfer vector according to claim 34, wherein said vetor is capable of expressing a dominant negative IGF-I receptor deposited at CNCM on Nov. 10, 2004 under accession number 1-3316.

36. A gene transfer vector according to claim 34, wherein said vector is capable of expressing a functional IGF-I receptor deposited at CNCM on Nov. 10, 2004 under accession number I-3315.

37. A lentiviral vector according to claim 34, wherein said vector is obtained by transitory transfection in package cells with:
A plasmid (i) which can read the sequence of nucleotides selected from:
a sequence of nucleotides that codify the dominant non functional mutated form of the IGF-I receptor, and
a sequence of nucleotides that codify an inhibitor element for IGF-I receptor gene expression capable of eliminating functional activity:
A plasmid (ii) that includes the sequence of nucleotides which codify the Rev protein;
A plasmid (iii) that includes the sequence of nucleotides that codify the Rev response element (RRE); and
A plasmid (iv) that includes the sequence of nucleotides that codify the heterogeneous vector casing.

38. The vector according to claim 37, wherein said plasmid (i) is a plasmid that can read the sequence of nucleotides that codify the non functional mutated form of the IGF-I receptor selected form a sequence of nucleotides that codify the non functional mutated for of the IGF-I receptor referred to as IGF-IR.KR which presents the mutation K1003R, where the lysine residue in position 1003 of the sequence of amino acids for the IGF-I human receptor has been substituted for arginine residues and the nucleotide sequence that codifies the non functional mutated form of the IGF-I receptor referred to as IGF-IR.KR showing the K1 003A mutation, in which the lysine residue in position 1003 of the amino acid sequence for the human IGF-I receptor has been substituted for an alanine residue.

39. The vector according to claim 37 wherein the plasmid (ii) is a plasmid that can read the sequence of nucleotides that codify an inhibitor element for IGF-I receptor gene expression capable of eliminating functional activity between a sequences of nucleotides that codify: a) an antisense nucleotide sequence specific to the gene sequence or to the IGF-I receptor mRNA, b) a ribozyme specific to the IGF-I receptor mRNA, c) a specific aptamer for the IGF-I receptor mRNA and d) RNA interference (RNAi) specific to the IGF-I receptor mRNA.

40-50. (canceled)

51. A method for treating or preventing a disease where abnormal brain accumulation of [beta] amyloid and/or amyloid plaques are involved in a mammal, wherein said method comprises administering to said mammal a molecule capable of increasing [beta] amyloid clearance from brain.

52. The method according claim 51, wherein said molecule promotes the entrance of a protein acting as a carrier of [beta] amyloid through the choroid plexus into the cerebrospinal fluid.

53. The method according to claim 52, wherein said carrier is albumin.

54. The method according to claim 52, wherein said carrier is transthyretin.

55. The method according to claim 52, wherein said carrier is apolipoprotein J.

56. The method according to claim 52, wherein said carrier is gelsolin.

57. The method according to claim 51, wherein the clearance of [beta] amyloid is increased by increasing the activity of IGF-I receptor in choroid plexus epithelial cells.

58. The method according to claim 57, wherein the molecule which is administered to the animal for increasing said IGF-I receptor activity is a gene transfer vector capable of inducing the expression of IGF-I receptor in target cells.

59. The method according to claim 58, wherein said gene transfer vector is derived from HIV or AAV.

60. The method according to claim 59, wherein said vector was deposited at CNCM on Nov. 10, 2004 under accession number I-3315.

61. Method of use of the nucleotide sequence encoding the IGF-I receptor for the prevention or treatment of a disease where abnormal brain accumulation of [beta] amyloid and/or amyloid plaques are involved, wherein said method involves administering said nucleotide sequence.

62. The method of use according to claim 61, wherein said disease is Alzheimer's disease.

63. Method of use of a nucleotide sequence encoding a polypeptide having a function analogous to the function of the IGF-I receptor, for the prevention or the treatment of a disease where abnormal brain accumulation of [beta] amyloid and/or amyloid plaques are involved wherein said method involves administering said nucleotide sequence.

64. Method of use according to claim 63, wherein the nucleotide sequence encodes an active fragment of the IGF-I receptor.

65. A therapeutic composition comprising a nucleotide sequence encoding a polypeptide having an analogous function to the function of the IGF-I receptor.

66. A therapeutic composition according to claim 65, wherein the nucleotide sequence encodes an active fragment of the IGF-I receptor.

67. A therapeutic composition which comprises the pHIV-IGFI R vector.
